(19) United States
(12) Patent Application Publication
(10) Pub. No.: US 2019/0290815 A1
(43) Pub. Date: Sep. 26, 2019

(54) Device, System and Method to Customize Chest Drainage Therapy

(71) Applicant: Alberto Antonicelli, Saronno (IT)

(72) Inventor: Alberto Antonicelli, Saronno (IT)

(21) Appl. No.: 16/082,091

(22) PCT Filed: Mar. 7, 2017
(86) PCT No.: PCT/US2017/021155

§ 371 Date: Sep. 4, 2018

Related U.S. Application Data
(60) Provisional application No. 62/304,822, filed on Mar. 7, 2016.
(60) Provisional application No. 62/343,031, filed on May 30, 2016.

(51) Int. Cl. A61M 1/04; A61M 1/00

(52) U.S. Cl. CPC A61M 1/04; A61M 1/0062; A61M 1/006; A61M 1/0035; A61M 2205/3334; A61M 2209/02; A61M 2210/101; A61M 2206/10; A61M 2205/3306; A61M 1/0001

(57) ABSTRACT

A device, system and method for characterizing a chest drainage apparatus. The device includes a source of both positive pressure and negative pressure, a conduit to provide the positive pressure and the negative pressure to the chest drainage apparatus and a sensor to detect a response of said chest drainage apparatus to either said positive pressure or said negative pressure. The system includes a device that controllably provides either a positive pressure or a negative pressure to the chest drainage apparatus and a sensor to record the effect of the positive pressure or the negative pressure to the drainage apparatus. The method includes the steps of (1) providing a source of both positive pressure and negative pressure; (2) controllably applying either the positive pressure or the negative pressure to the chest drainage apparatus; and (3) detecting a response from the chest drainage apparatus to the pressure application.

This patent application claims priority to U.S. Provisional Patent Application Ser. No. 62/304,822, titled “System and Device to Protect Patients from Excessive Negative Intrapleural Pressures,” by Alberto Antonicelli, that was filed on Mar. 7, 2016. This patent application also claims priority to U.S. Provisional Patent Application Ser. No. 62/343,031, titled “Automatic Pilot to Tailor Chest Drainage Therapy to Individual Patients,” by Alberto Antonicelli, that was filed on May 30, 2016. The disclosures of both U.S. 62/304,822 and U.S. 62/343,031 are incorporated by reference herein in their entireties.

A chest tube is required in most patients diagnosed with and treated for thoracic diseases. The chest tube removes air and fluids (such as serous fluid, blood, chyle or pus) from the pleural space. A representative chest drainage apparatus is disclosed in U.S. Pat. No. 4,738,671, titled “Chest Drainage Apparatus with Check Valve,” by Elliott, et al. Key features of this chest drainage system include a connecting tube that connects a proximal end of a chest tube to a collection chamber. An opposing distal end of the chest tube is inserted into a patient's pleural space for removal of air and fluid. A second tube connects the collection chamber to a vacuum source that applies a negative pressure to the chest drainage apparatus through the collection chamber to further promote the flow of air and fluids from the pleural space to the collection chamber. This externally applied negative pressure must be carefully regulated. If too low, the patient may draw both air and fluids back to the pleural space during inspiration, when one-bottle chest drainage systems are used; or only air when two-bottle, three-bottle or compact chest drainage systems are used. If the externally applied negative pressure is too high, structures within the chest cavity may be damaged, particularly when the patient is in a fragile state. U.S. Pat. No. 4,738,671 is incorporated by reference in its entirety herein.

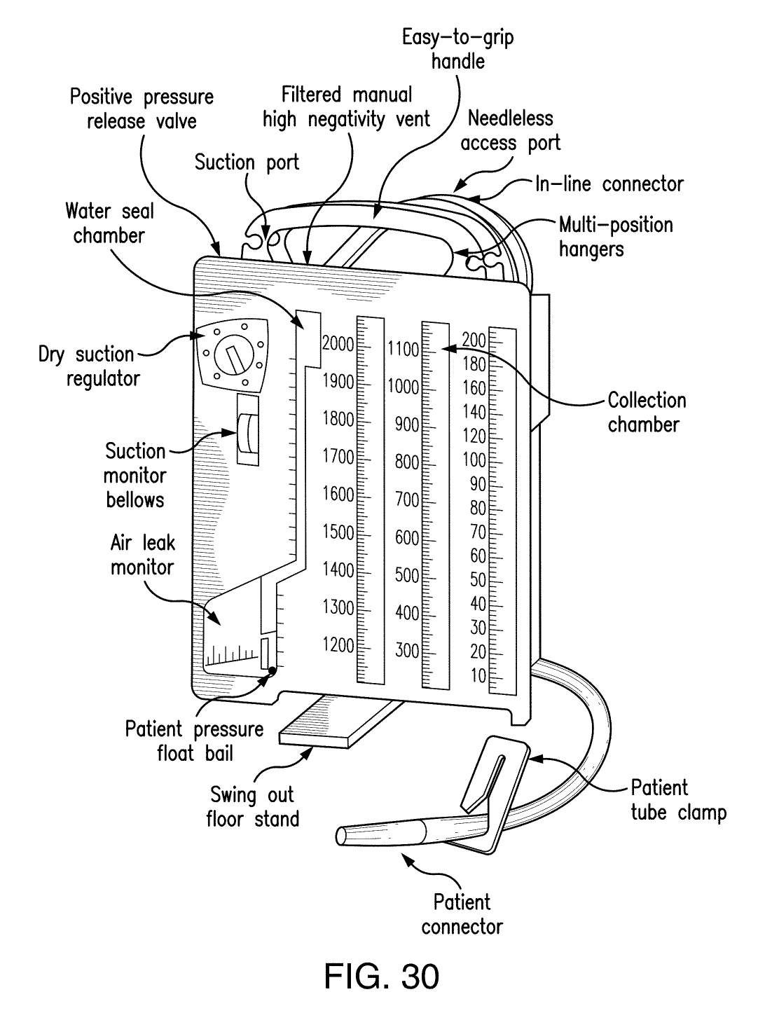

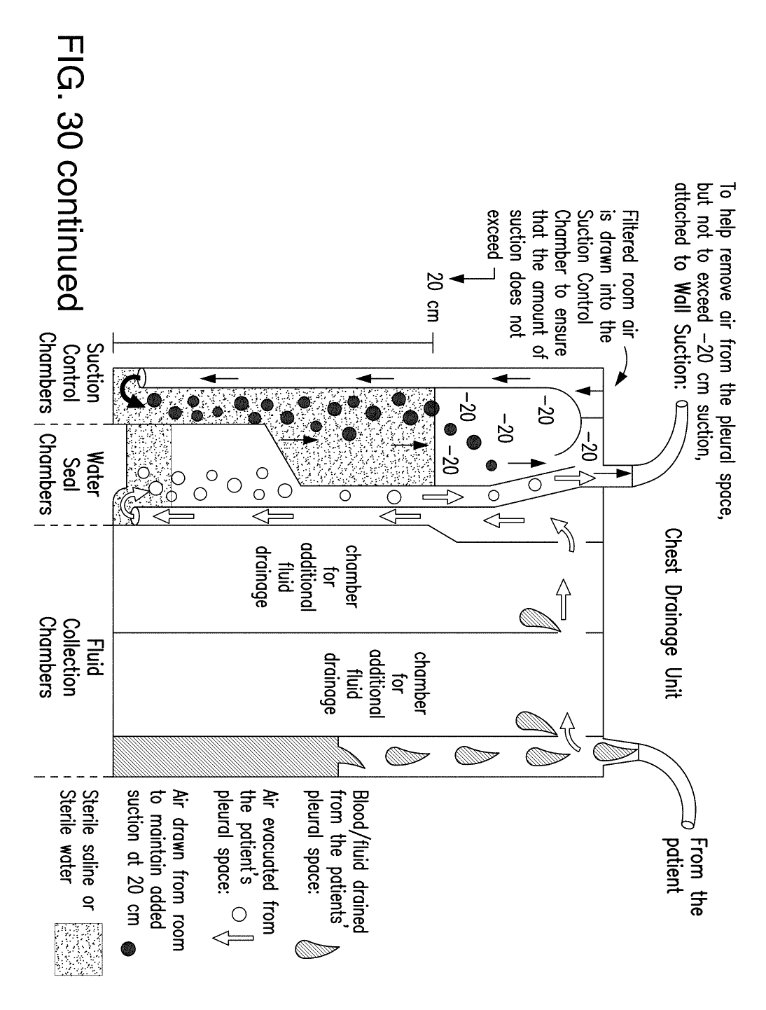

A generic chest drainage system as known from the prior art is shown in FIG. 30. Chest drainage systems are in themselves therapeutic for the ongoing disease. Optimizing management of pleural drainage promotes patient recovery. However, the pathophysiology of the pleural space has been considered an obscure topic for decades and true investigation of the science is relatively sparse. Most surgeons are taught, and accept, dogmas laid down by empirical observations and passed on from one generation to the next. There is a lack of proper regulations and standards governing chest drainage systems' technical evolution accompanied by an absence of clinical guidelines for chest drainage therapy. As a consequence, the evolution of chest drainage systems has not been done with any guidance. Indeed, device designs of currently available “traditional” chest drainage systems [Under-Water Sealed Drainage (UWSD) and dry seal], as well as principles by which software and hardware are engineered in digital systems, differ greatly from device to device and from manufacturer to manufacturer. At present, a large number of different types of chest drainage systems, estimated to exceed forty, are commercially available worldwide. Frequently, a doctor's exposure to more than one type of chest drainage system is limited by hospital administration preference based on price or preferred source.

For the foregoing reasons, current management of patients with chest tubes is unstandardized. Satisfactory treatment may result from evidence-based protocols in experienced hospitals, but the treatments are not necessarily up-to-date with scientific evidence. Overall heterogeneous, or even inadequate, clinical performance of certain chest drainage systems introduces biases in clinical trials, making it difficult to interpret and compare results, and making development of personalized treatments a great challenge.

Furthermore, thoracic surgical patients encompass a wider spectrum of diseases than what was previously considered feasible. For example, surgery is now offered more often to patient with end-stage fibrosis or emphysema. These are high-risk patients and, as such, should be considered for tailored chest drainage therapies. However, the market lacks a system capable to address this.

Pulmonary complications after cardiothoracic surgery increase mortality and morbidity, hospital length of stay and costs. Adequate drainage of the pleural space from air and fluid is a cornerstone of good management. While several chest drainage systems are commercially available worldwide, their performances vary and are suboptimal in certain patients. Furthermore, not only is true investigation of the principles by which they work sparse, but also, many doctors still rely on knowledge that is based on empirical observations rather than on substantial scientific evidence.

Preliminary work related to the nature of pleural space management and its consequences has been undertaken. Two laboratory investigations showed that different chest drainage system, having different performance parameters, set the stage for complications. See, for example, Alberto Antonicelli et al., Water Seal's One-Way Action in Chest Drainage Systems: When the Paradigm Fails (Copenhagen, 2014) and Alberto Antonicelli, Potentially Dangerous Negative Intrapleural Pressure: Pros and Cons of Digital Chest Drainage Systems (Naples 2016).

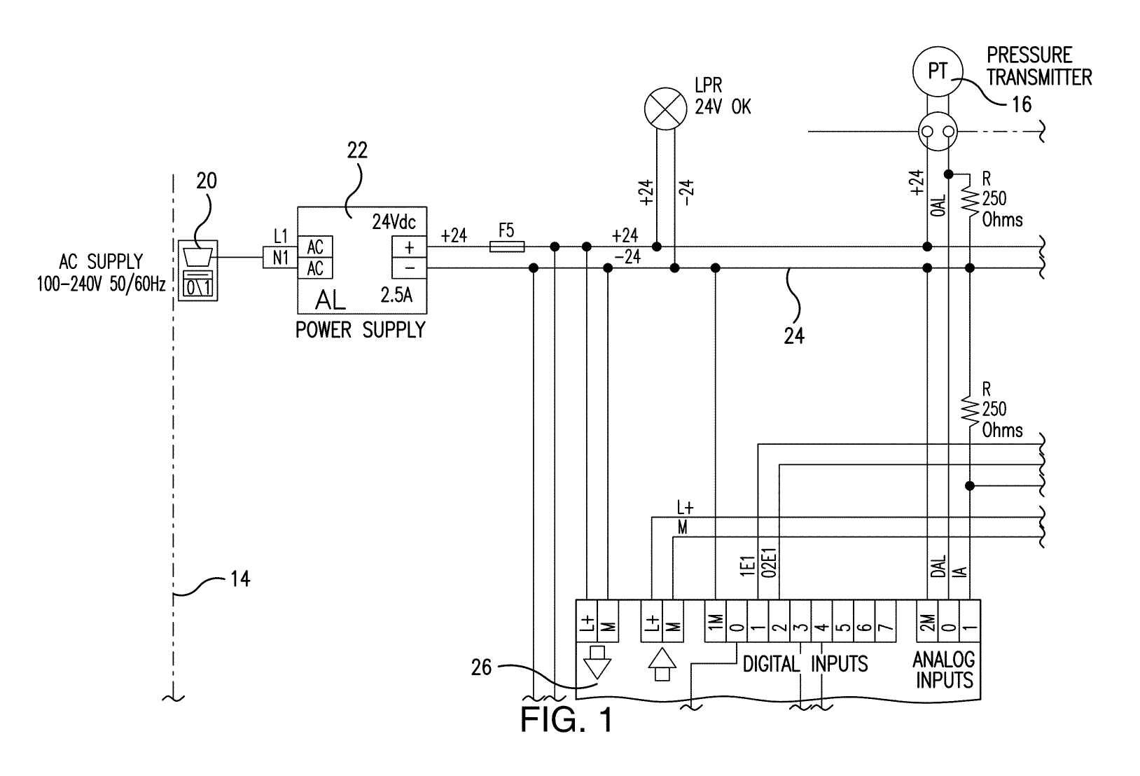

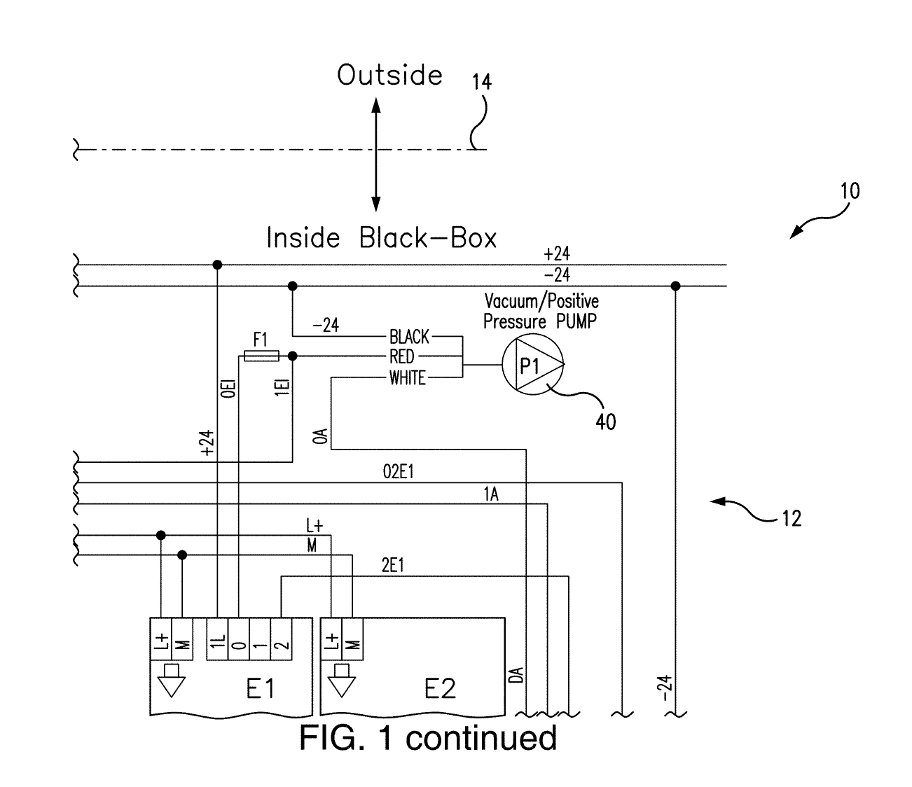

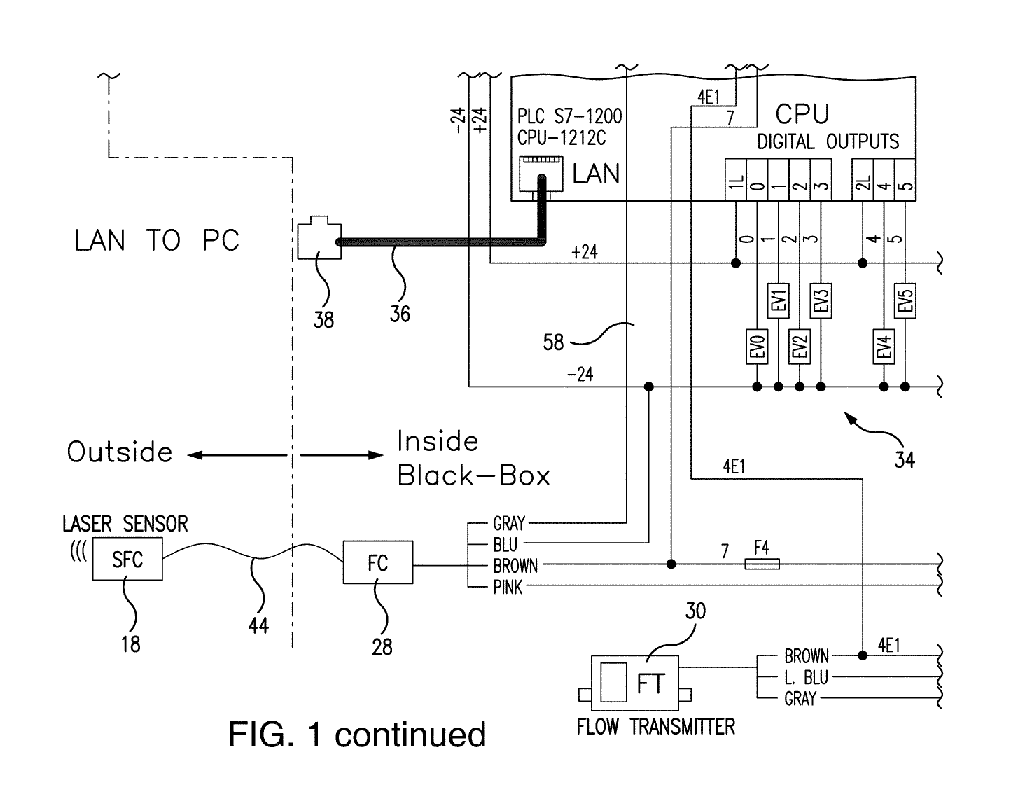

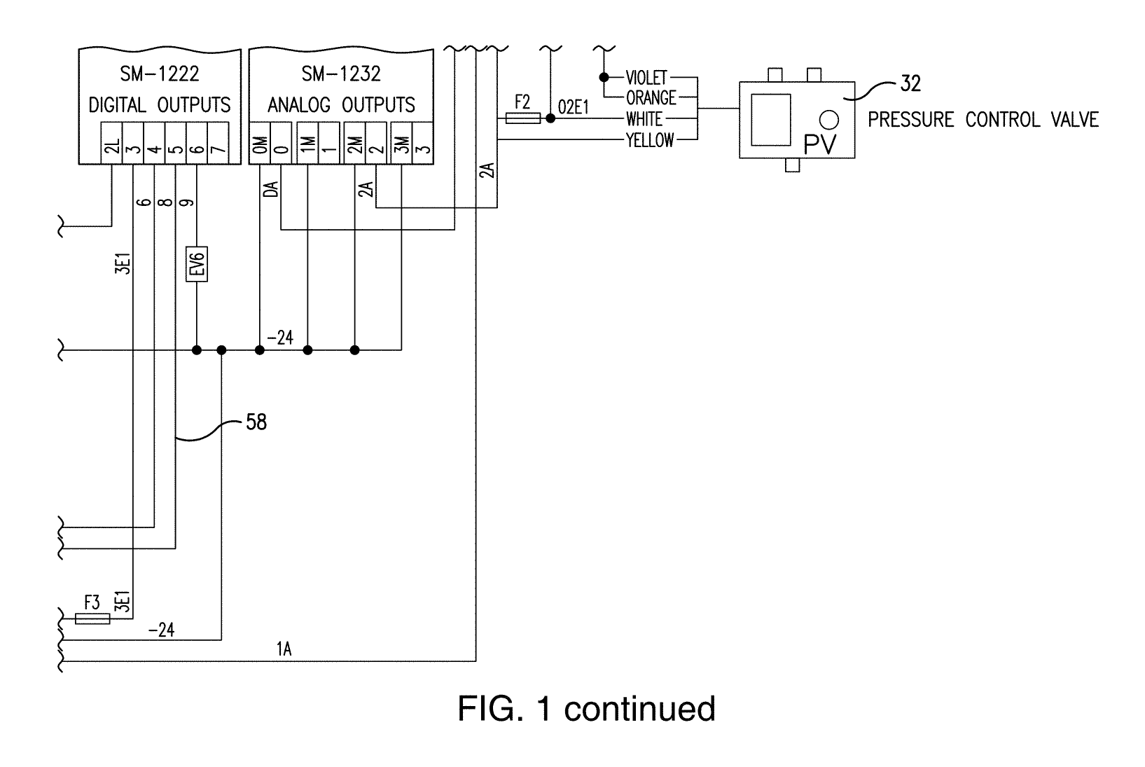

FIG. 1 schematically illustrates a device for characterizing a chest drainage system in accordance with the first embodiment herein.

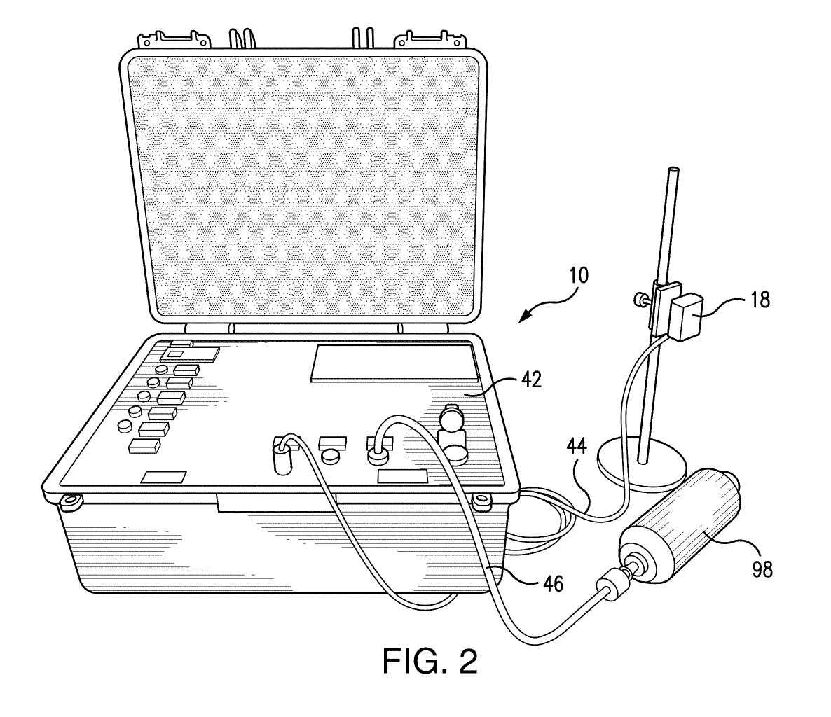

FIG. 2 is a photograph of the exterior of the device of FIG. 1 showing select external components.

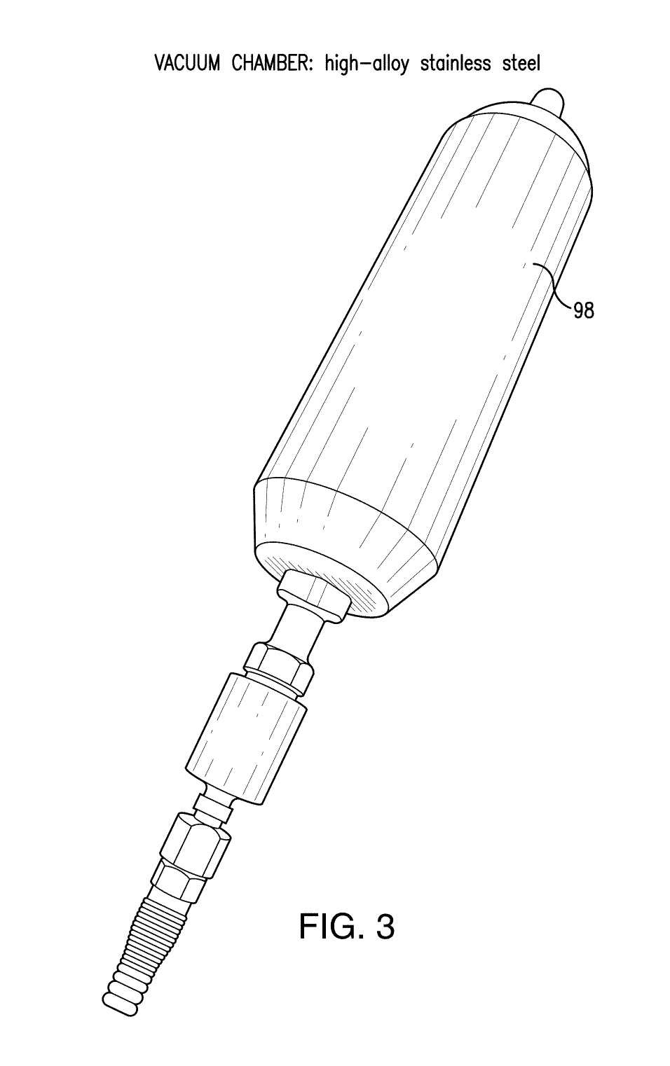

FIG. 3 is a photograph of a vacuum chamber as one of the external components of FIG. 2.

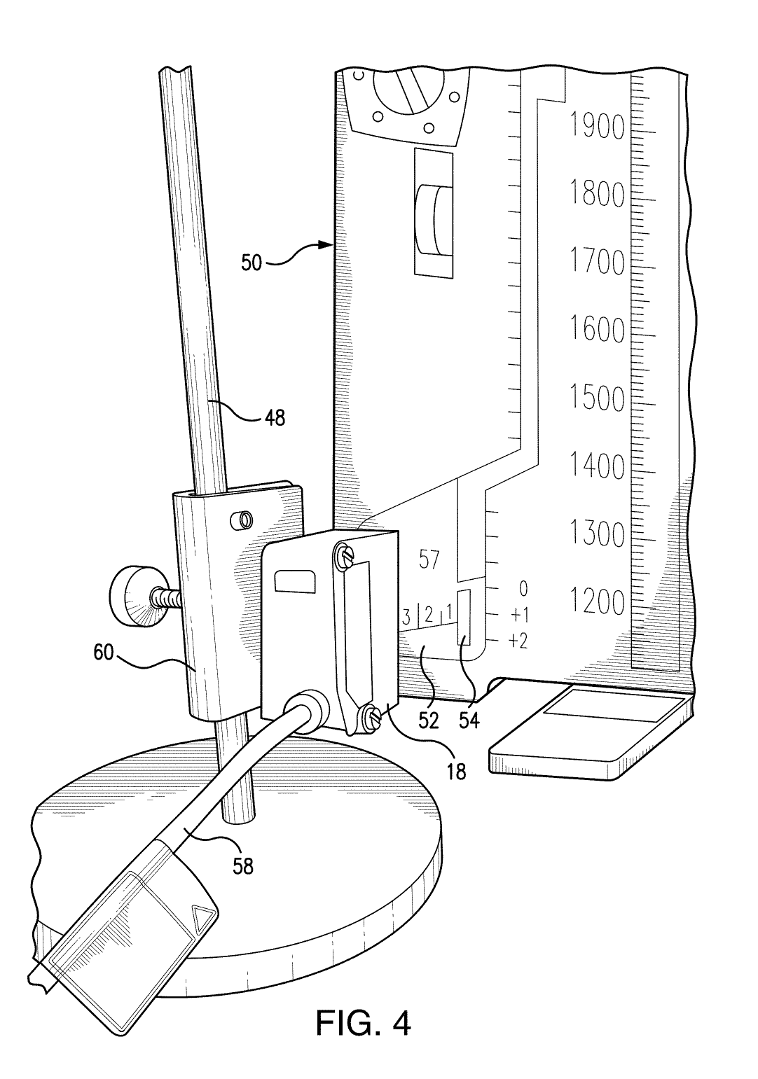

FIG. 4 is a photograph of a laser sensor as one of the external components of FIG. 2.

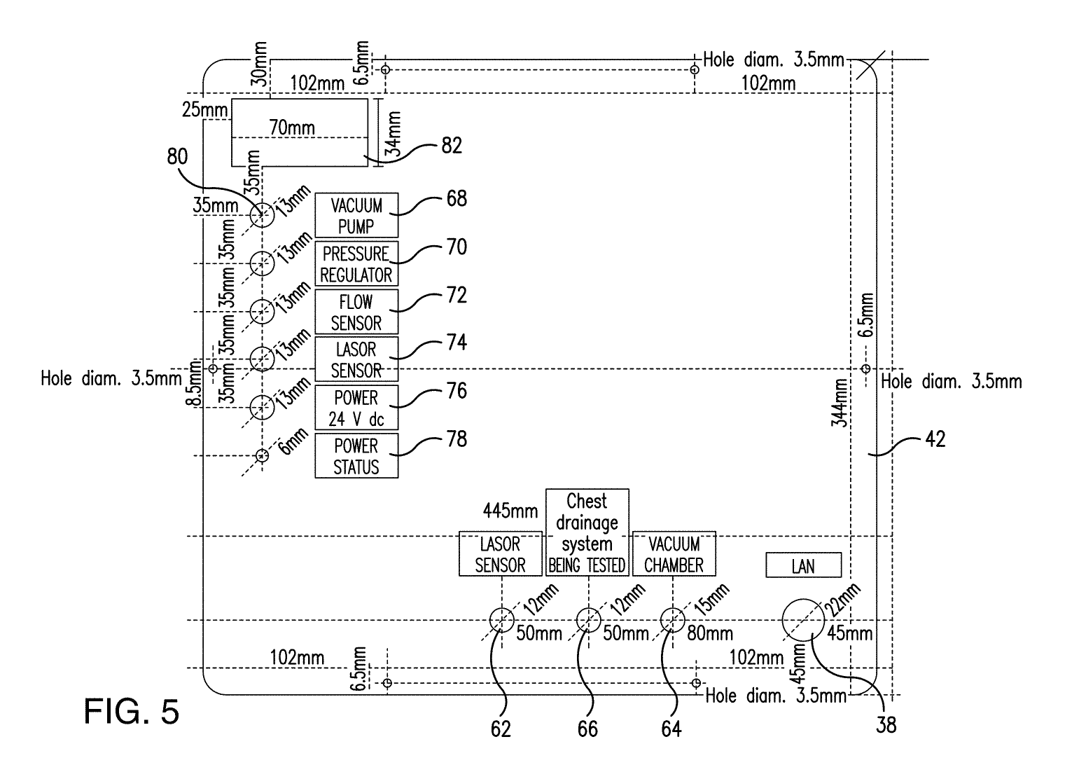

FIG. 5 schematically illustrates a control panel for the device of FIG. 1.

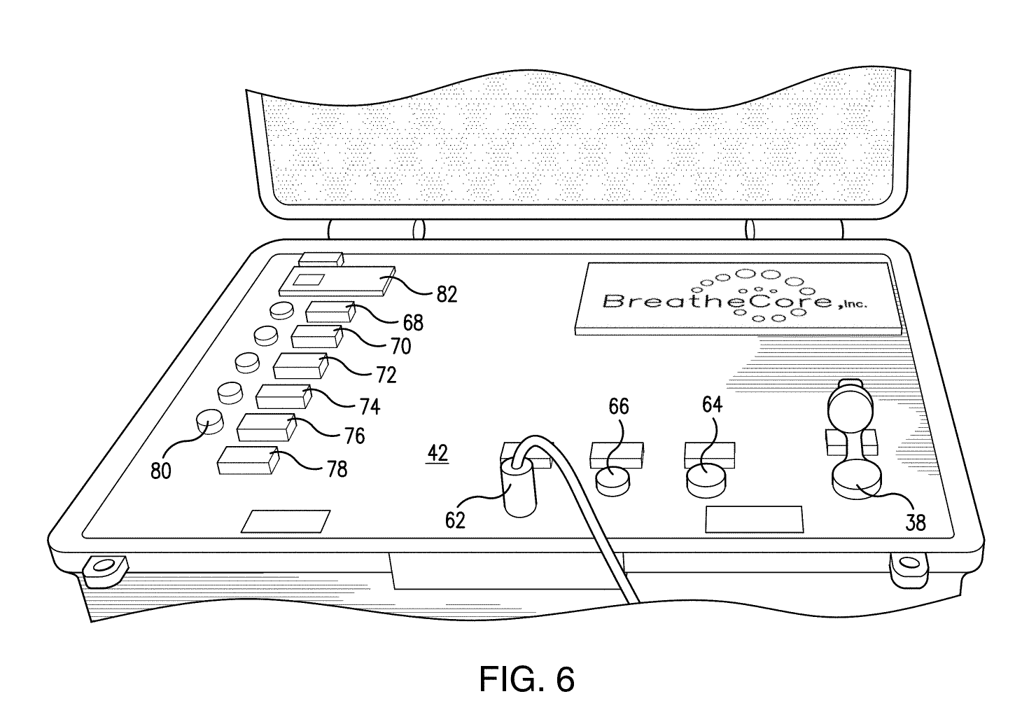

FIG. 6 is a photograph of the control panel of FIG. 5.

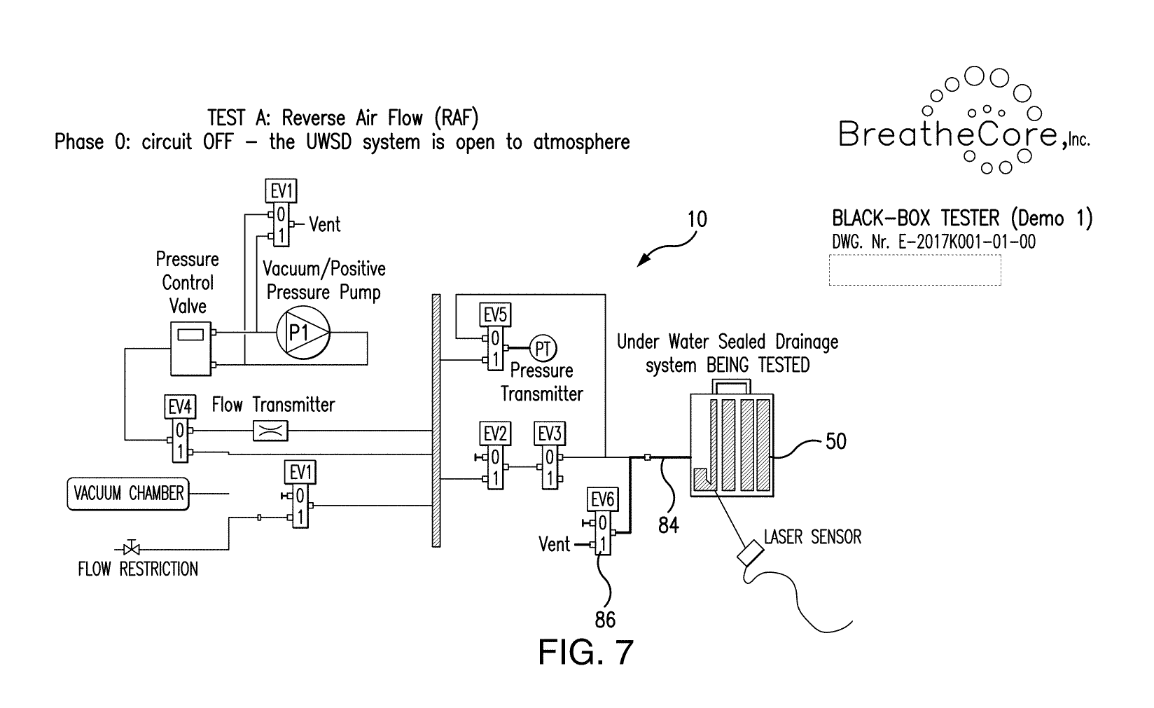

FIG. 7 diagrammatically illustrates a system to characterize an integrated, 3-bottle, UWSD system for a susceptibility to air retraction utilizing the device of FIG. 1 at a first step of the characterization process.

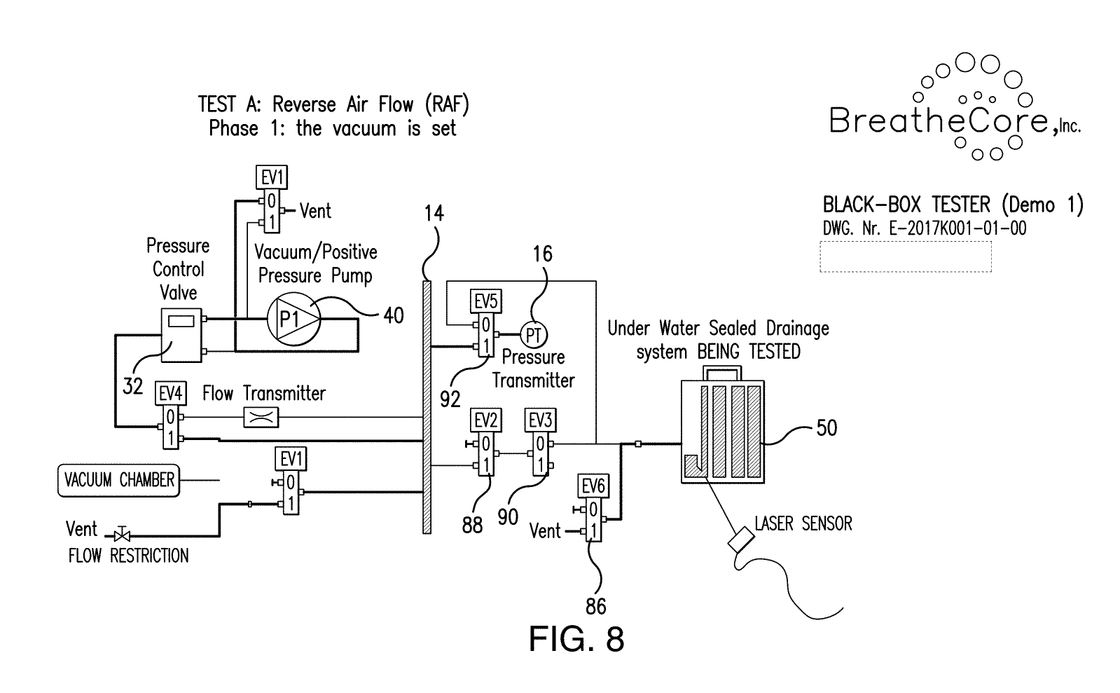

FIG. 8 diagrammatically illustrates the system of FIG. 7 at a second step of the characterization process.

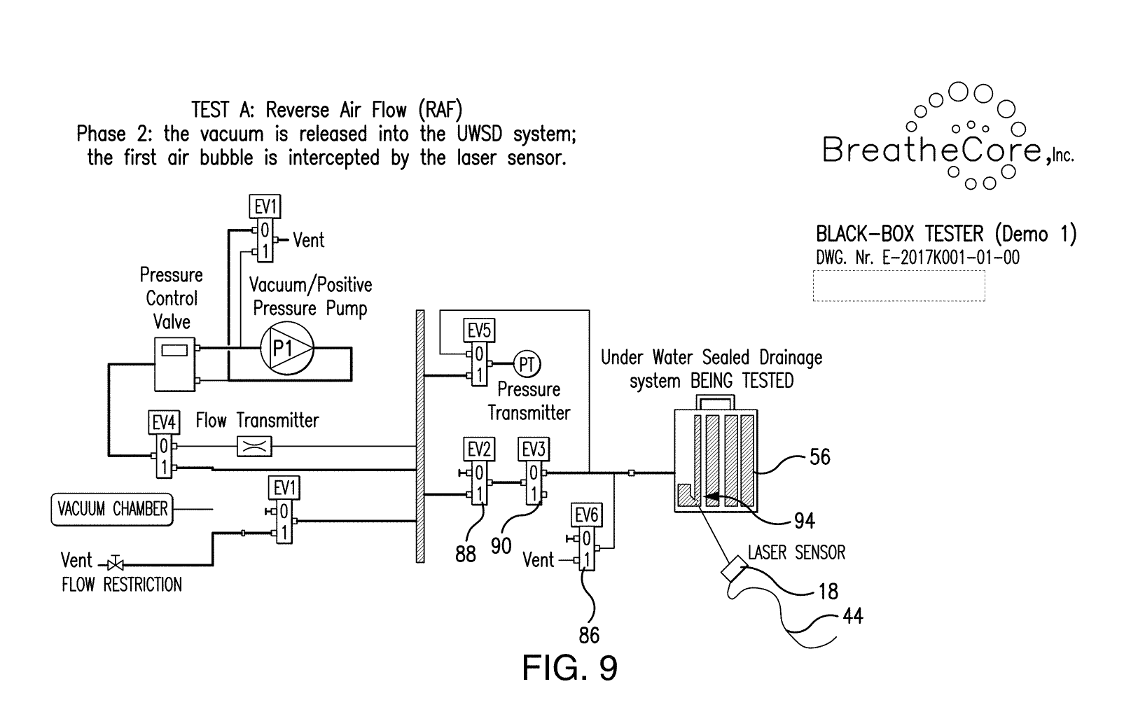

FIG. 9 diagrammatically illustrates the system of FIG. 7 at a third step of the characterization process.

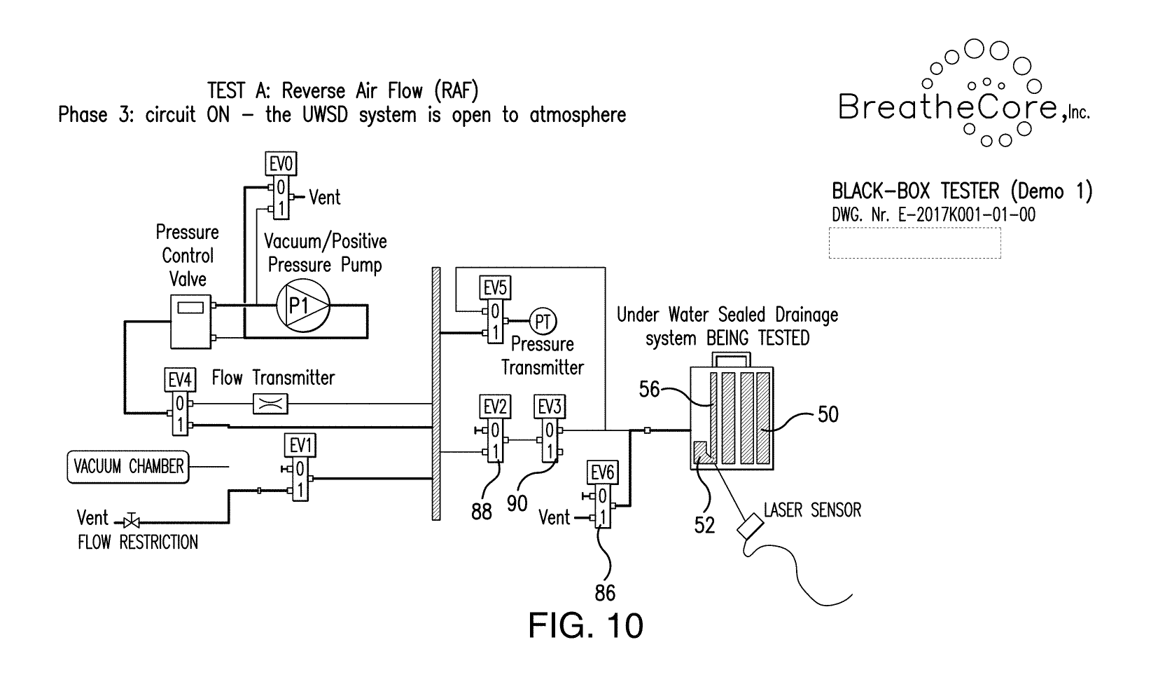

FIG. 10 diagrammatically illustrates the system of FIG. 7 at a fourth step of the characterization process.

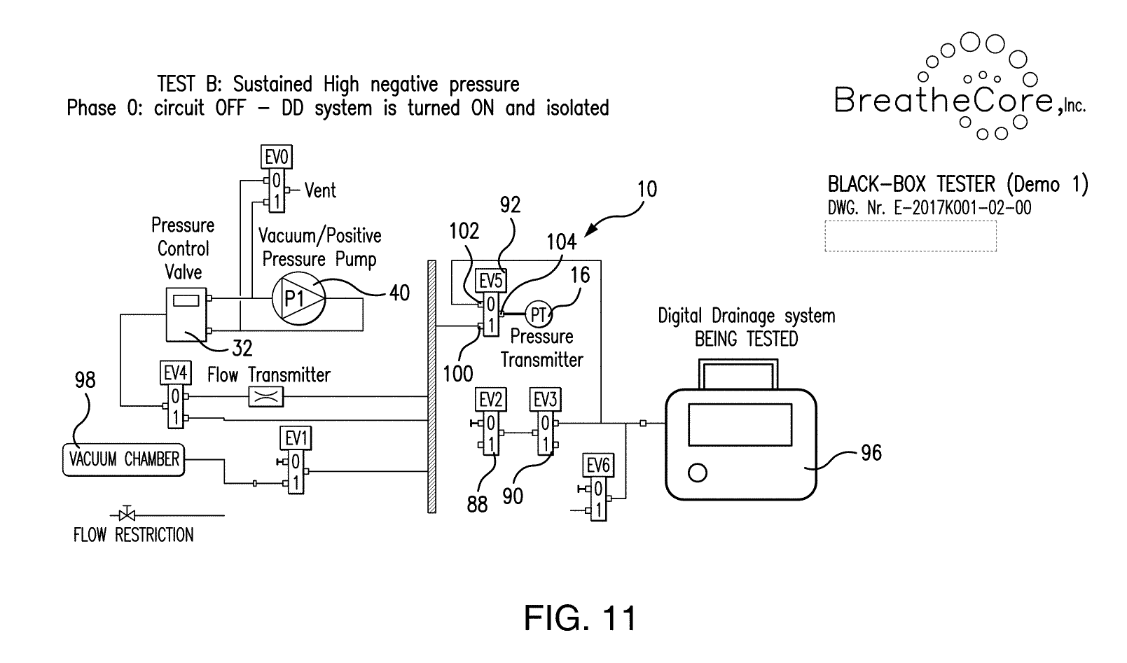

FIG. 11 diagrammatically illustrates a system to characterize a chest drainage system for the capability to handle high negative intrapleural pressure utilizing the device of FIG. 1 at a first step of the characterization process.

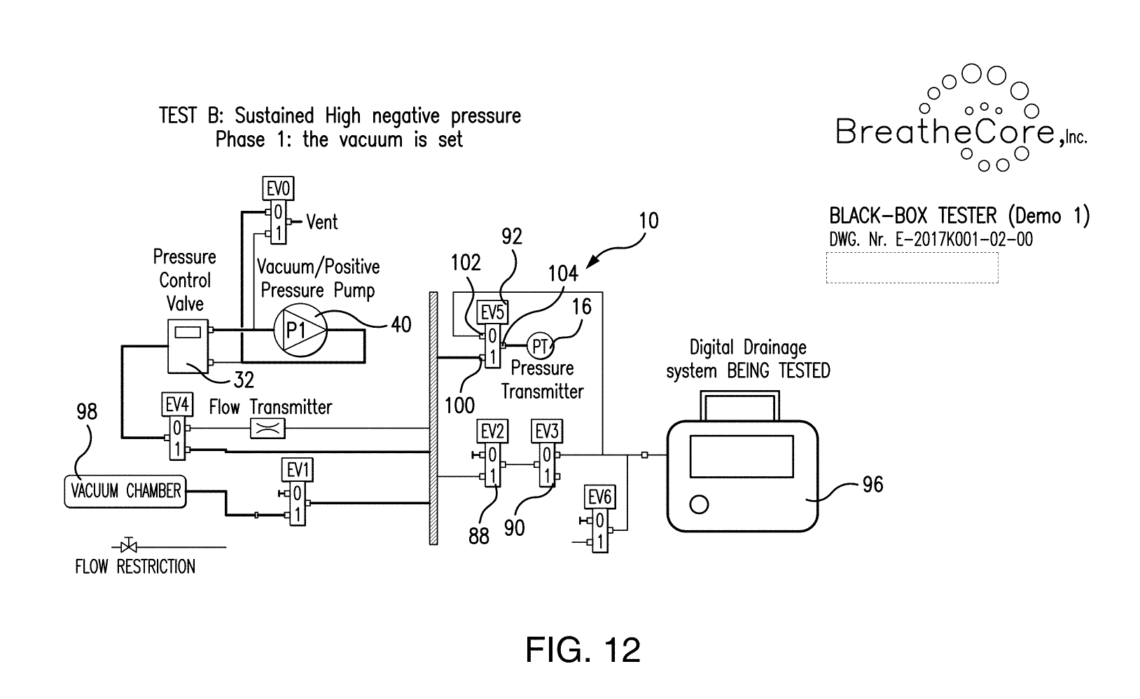

FIG. 12 diagrammatically illustrates the system of FIG. 11 at a second step of the characterization process.

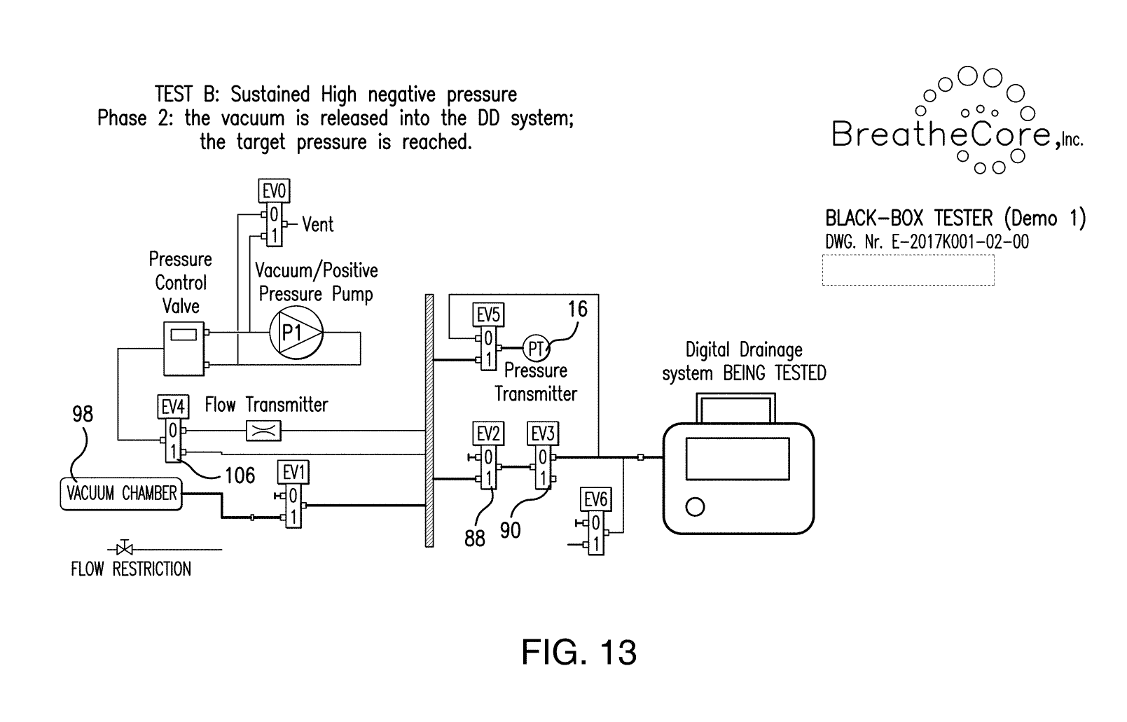

FIG. 13 diagrammatically illustrates the system of FIG. 11 at a third step of the characterization process.

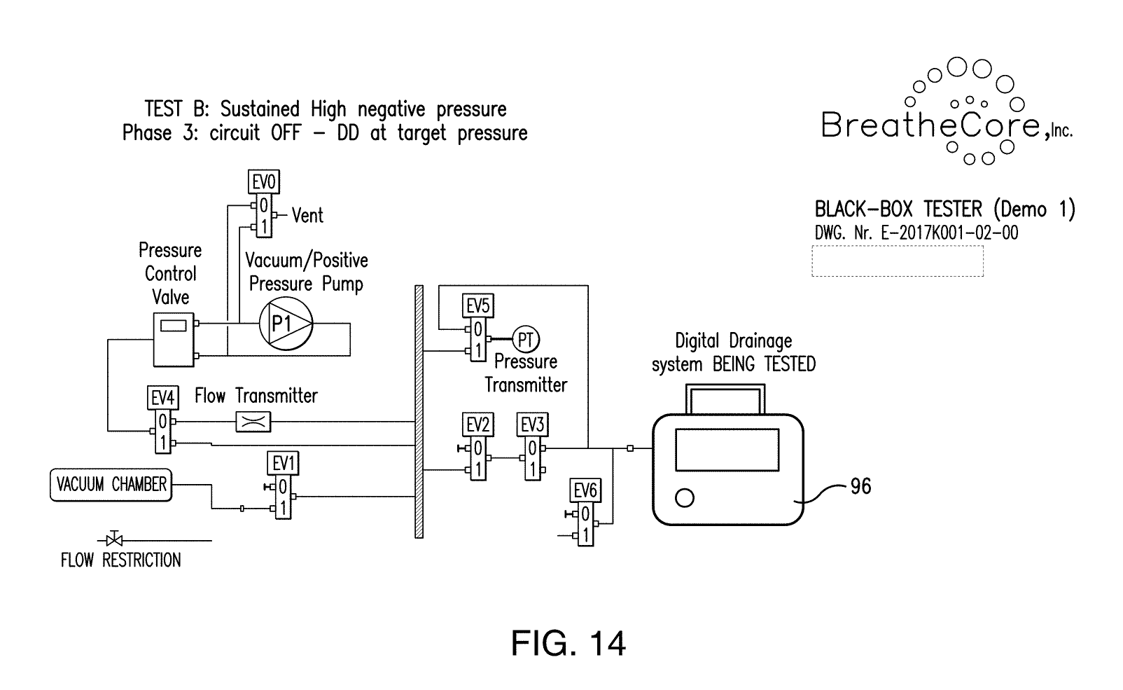

FIG. 14 diagrammatically illustrates the system of FIG. 11 at a fourth step of the characterization process.

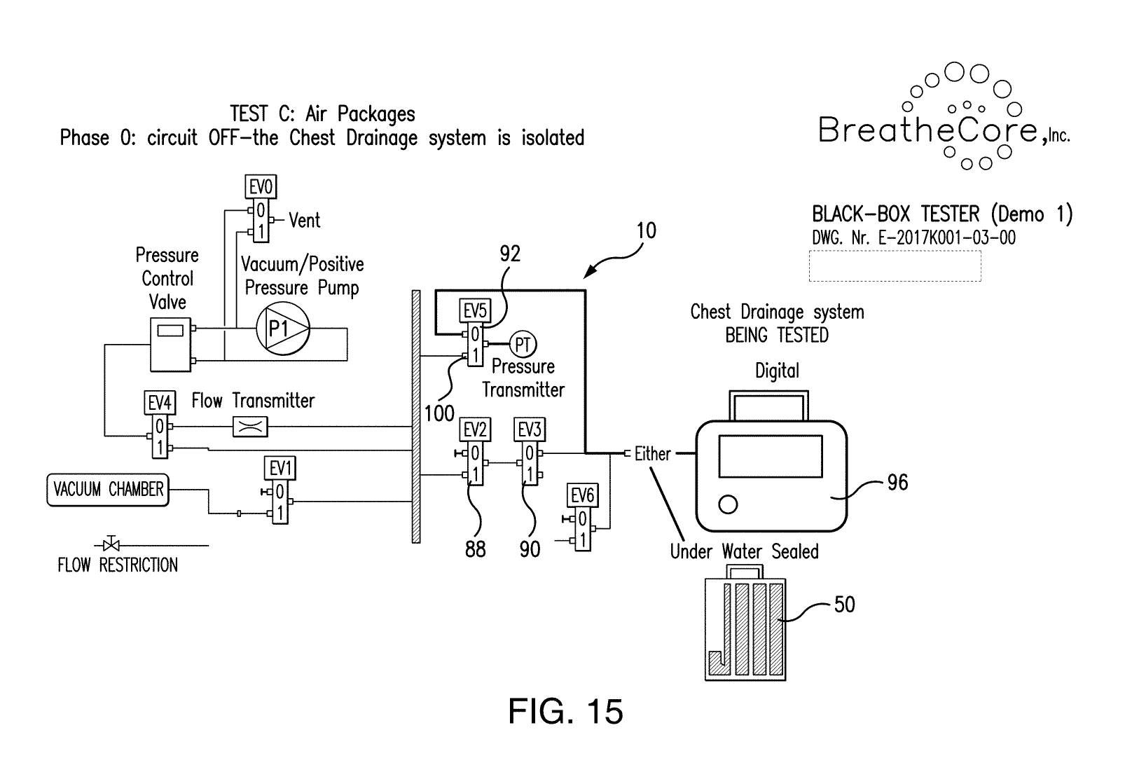

FIG. 15 diagrammatically illustrates a system to characterize a chest drainage system for the capability to handle a bronco-pulmonary air leak utilizing the device of FIG. 1 at a first step of the characterization process.

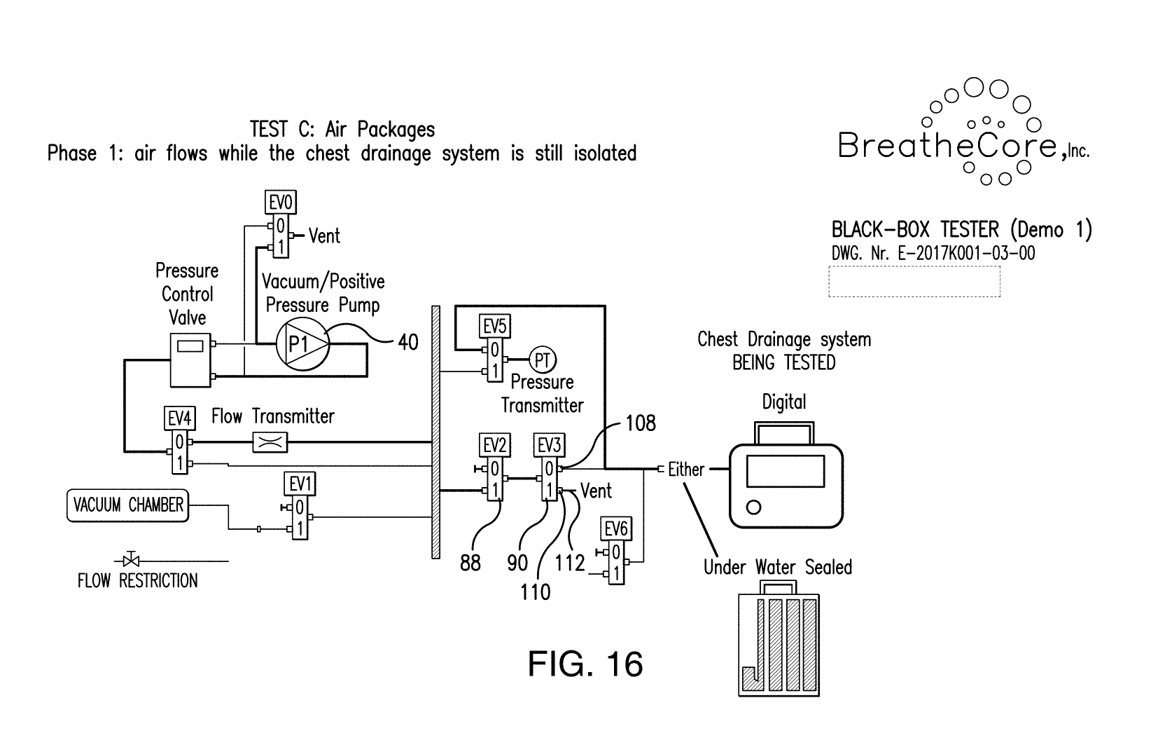

FIG. 16 diagrammatically illustrates the system of FIG. 15 at a second step of the characterization process.

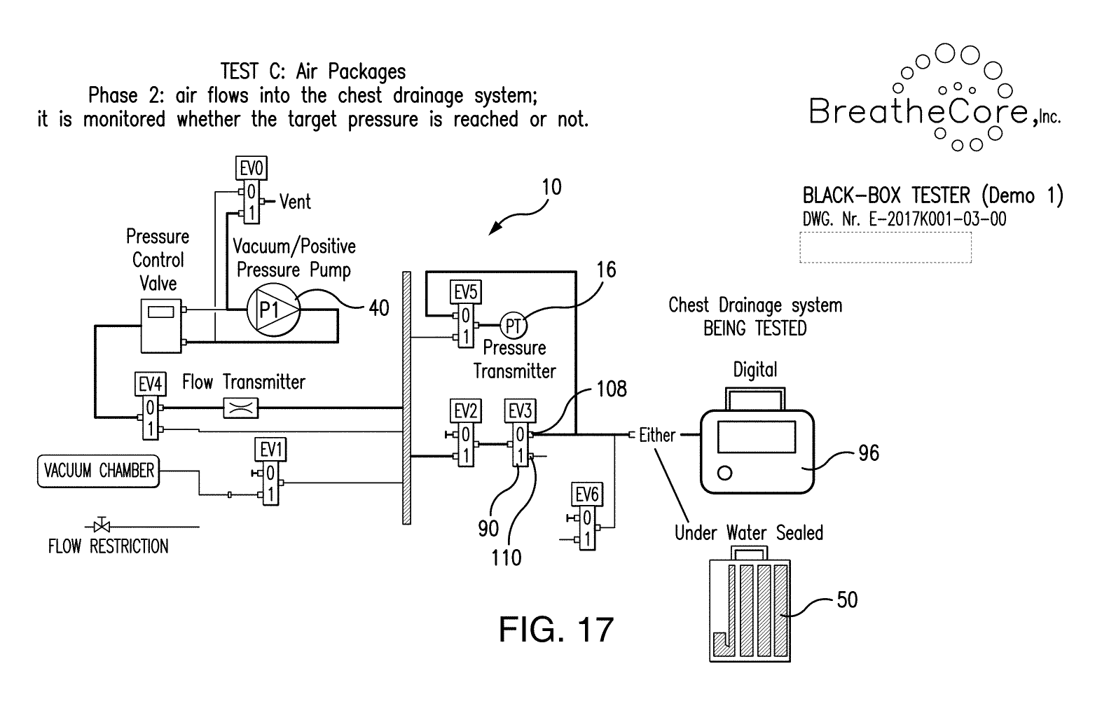

FIG. 17 diagrammatically illustrates the system of FIG. 15 at a third step of the characterization process.

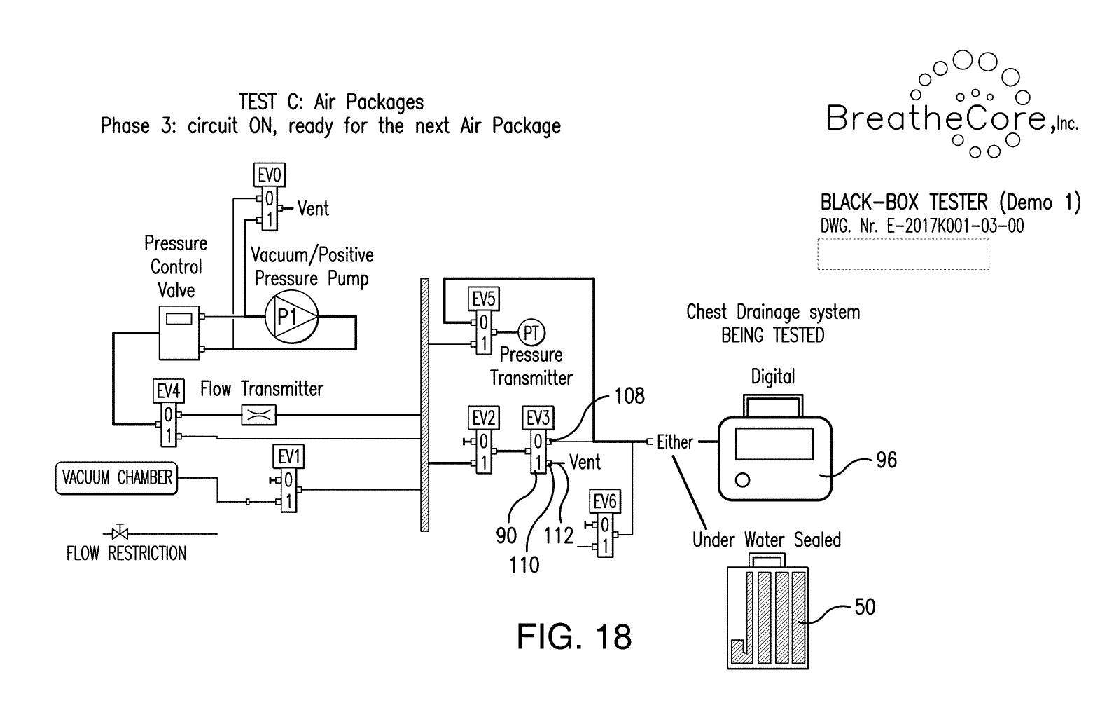

FIG. 18 diagrammatically illustrates the system of FIG. 15 at a fourth step of the characterization process.

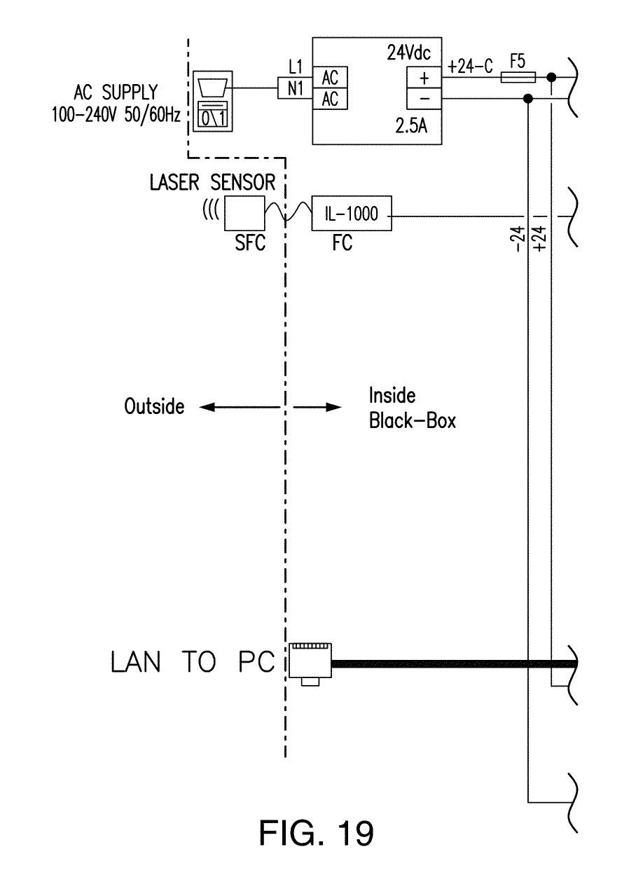

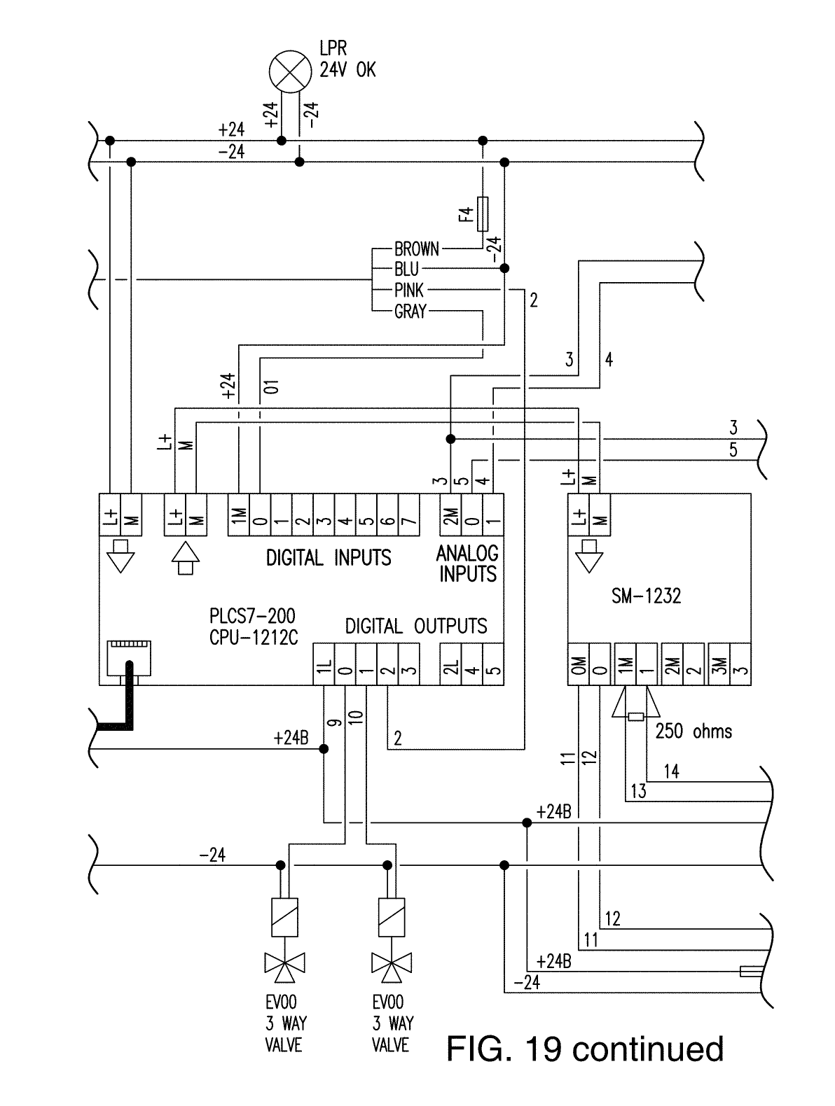

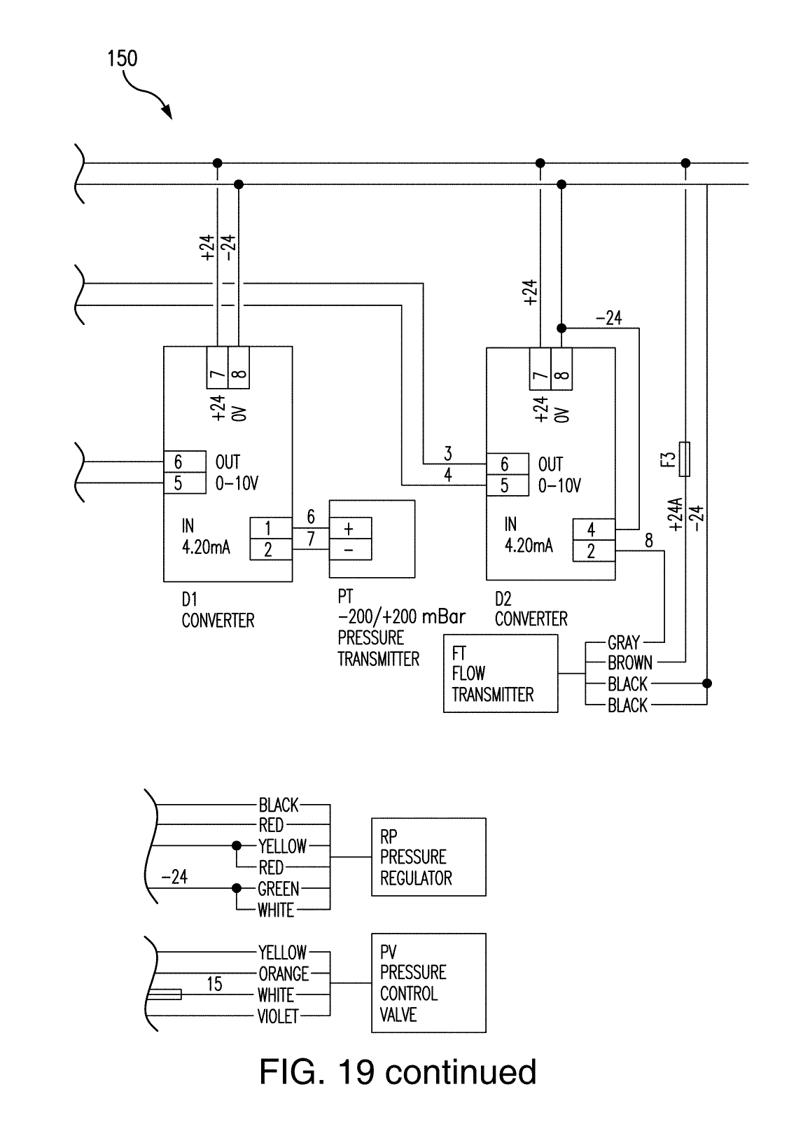

FIG. 19 schematically illustrates a device for characterizing a chest drainage system in accordance with the second embodiment herein.

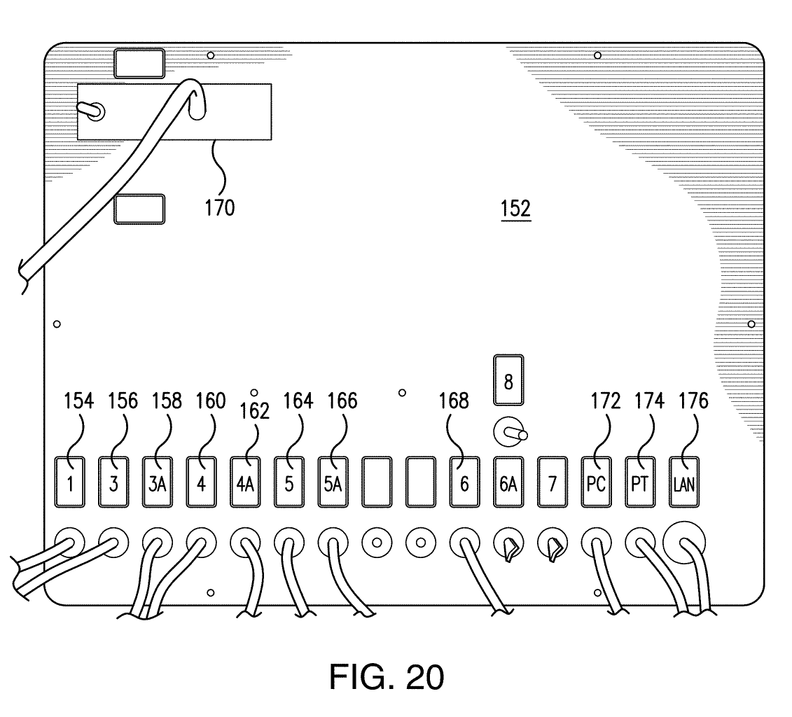

FIG. 20 is a photograph of a control panel for the device of FIG. 19.

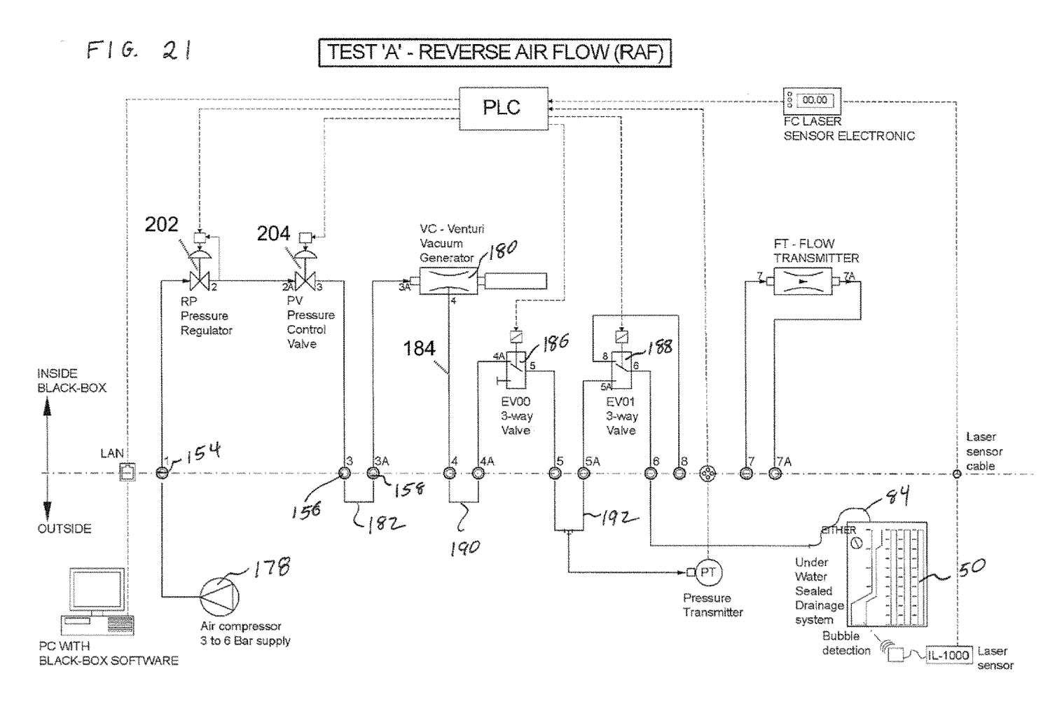

FIG. 21 diagrammatically illustrates a system to characterize a chest drainage system for a susceptibility to air retraction utilizing the device of FIG. 19.

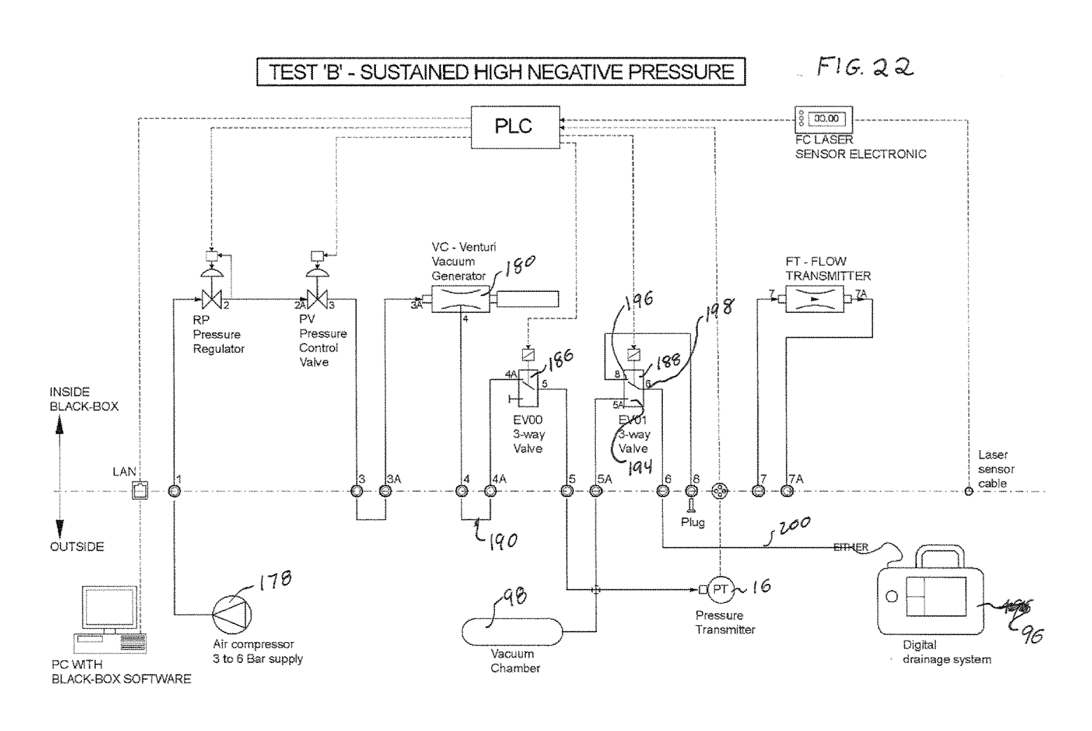

FIG. 22 diagrammatically illustrates a system to characterize a chest drainage system for the capability to handle high negative intrapleural pressure utilizing the device of FIG. 19.

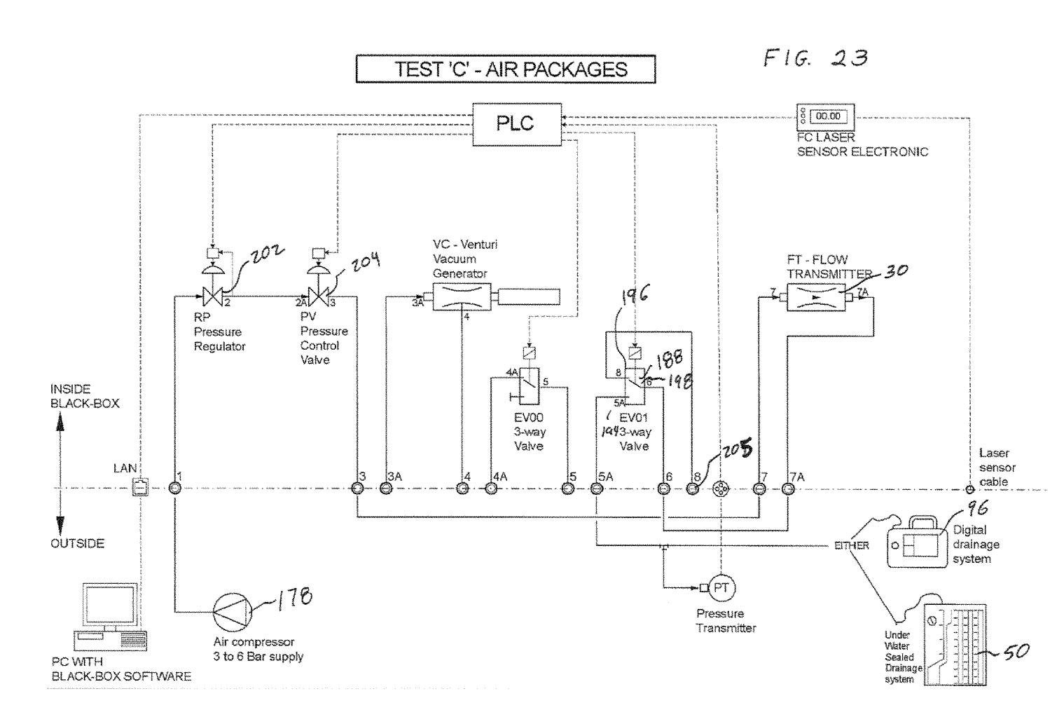

FIG. 23 diagrammatically illustrates a system to characterize a chest drainage system for the capability to handle a bronco-pulmonary air leak utilizing the device of FIG. 19.

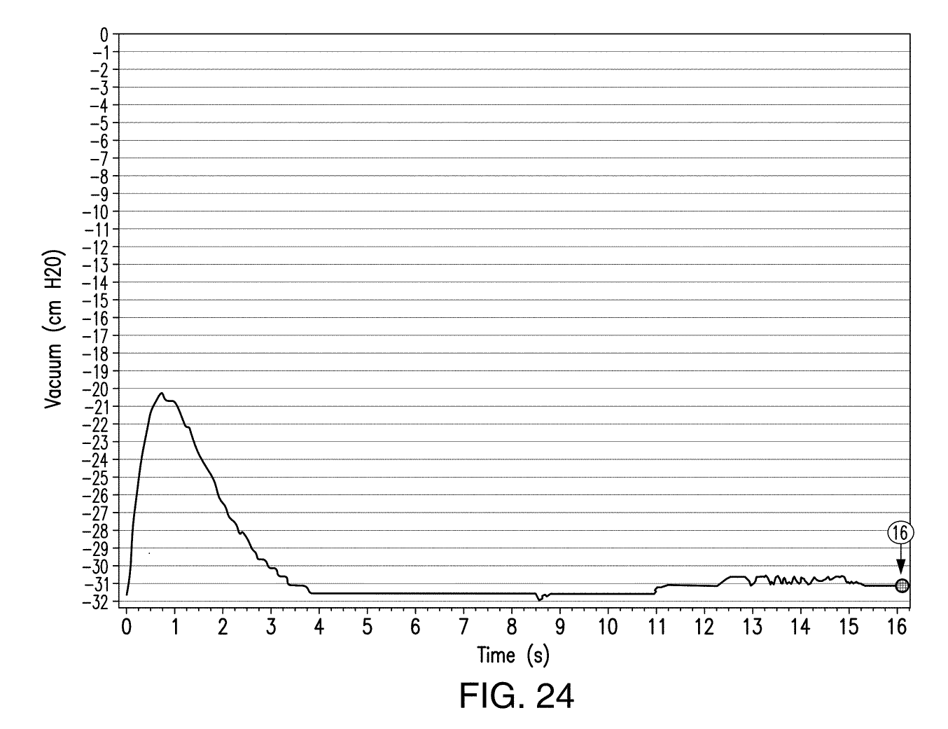

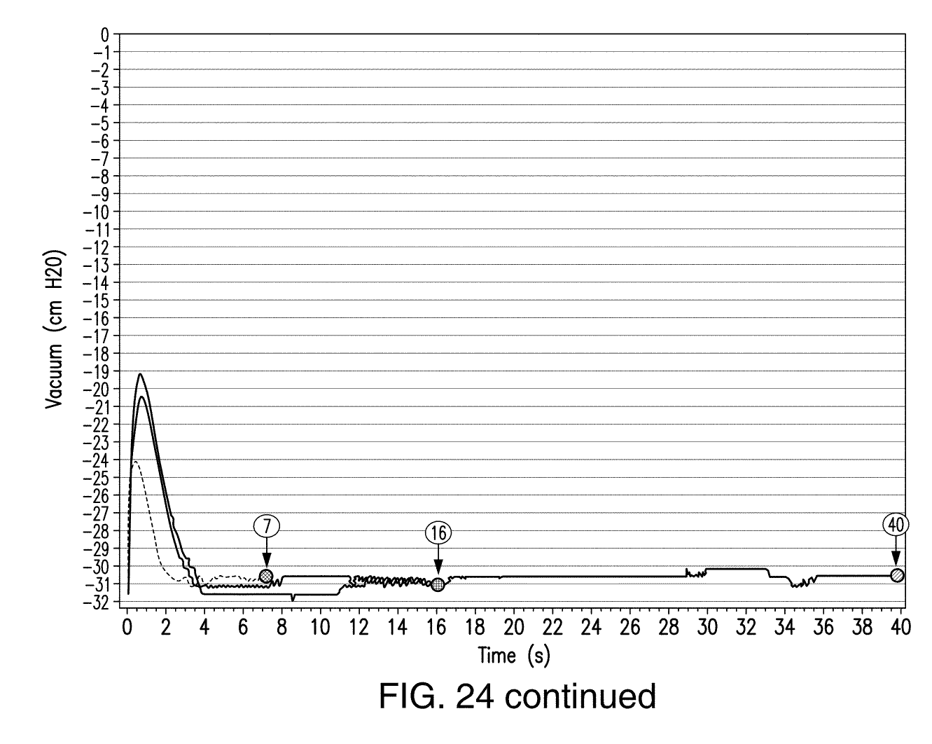

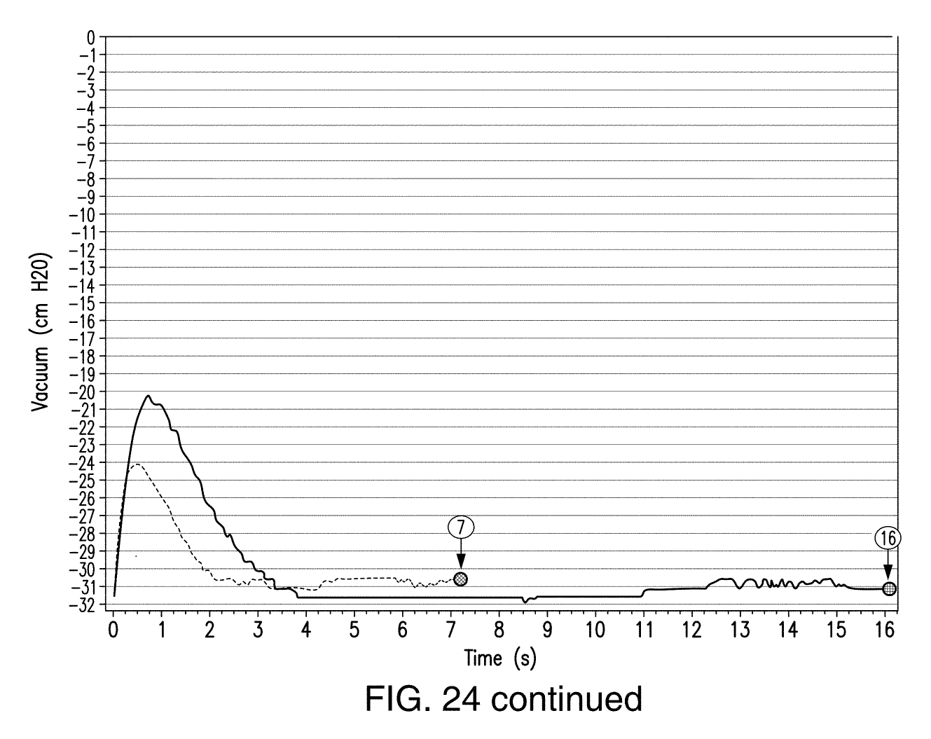

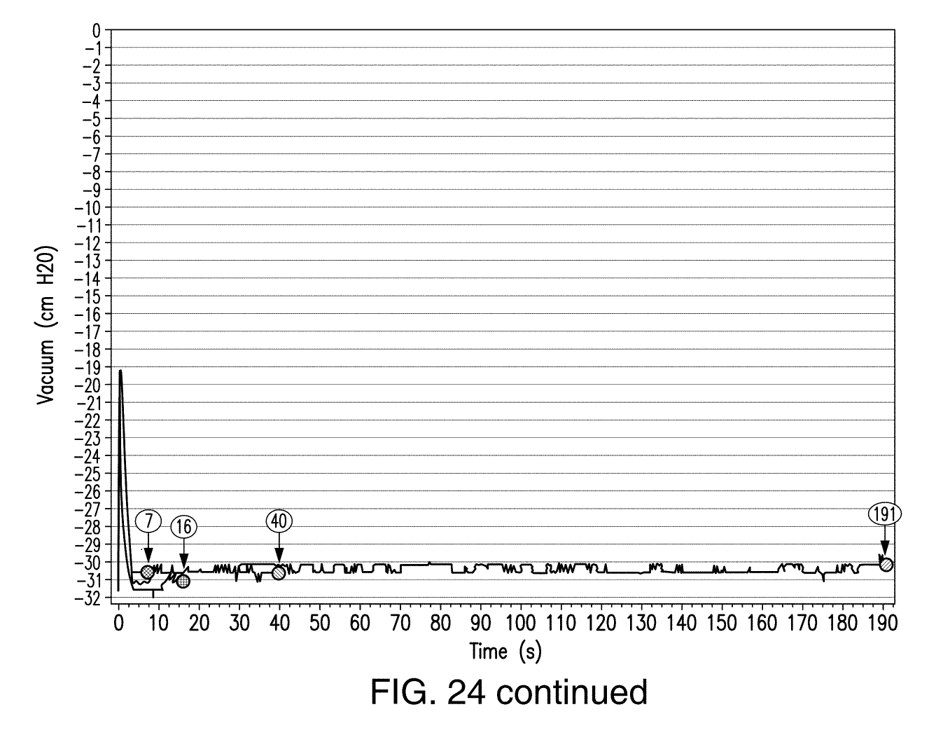

FIG. 24 is a sequential, scaled, graphical comparison of four integrated, 3-bottle, UWSD systems for susceptibility to air retraction.

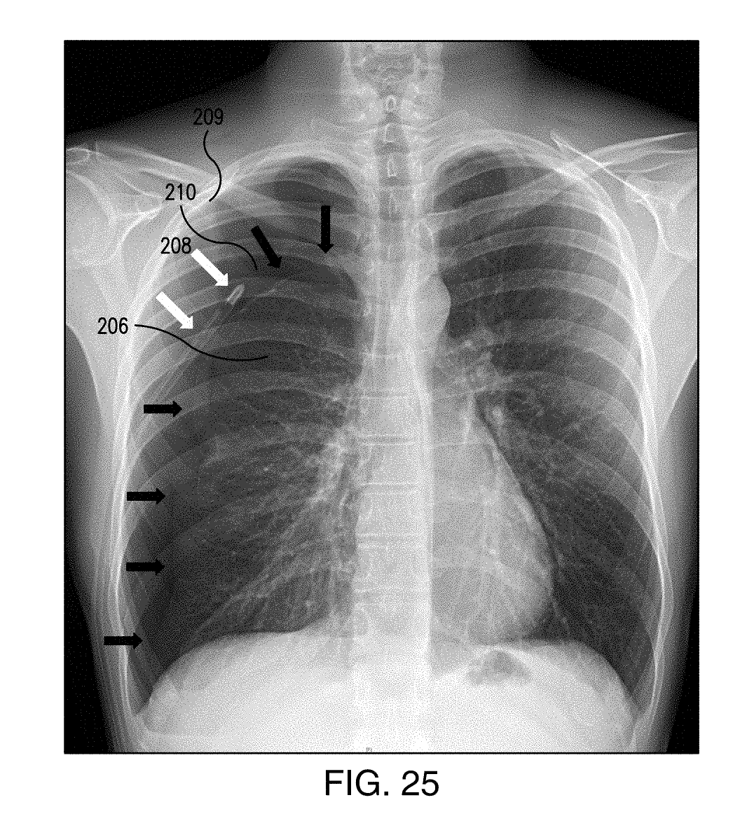

FIG. 25 is a chest X-ray showing the distal end of a chest tube inserted into a pleural space and the effect of an integrated, 3-bottle, UWSD system having unregulated and great susceptibility to air retraction (iatrogenic persistent pneumothorax).

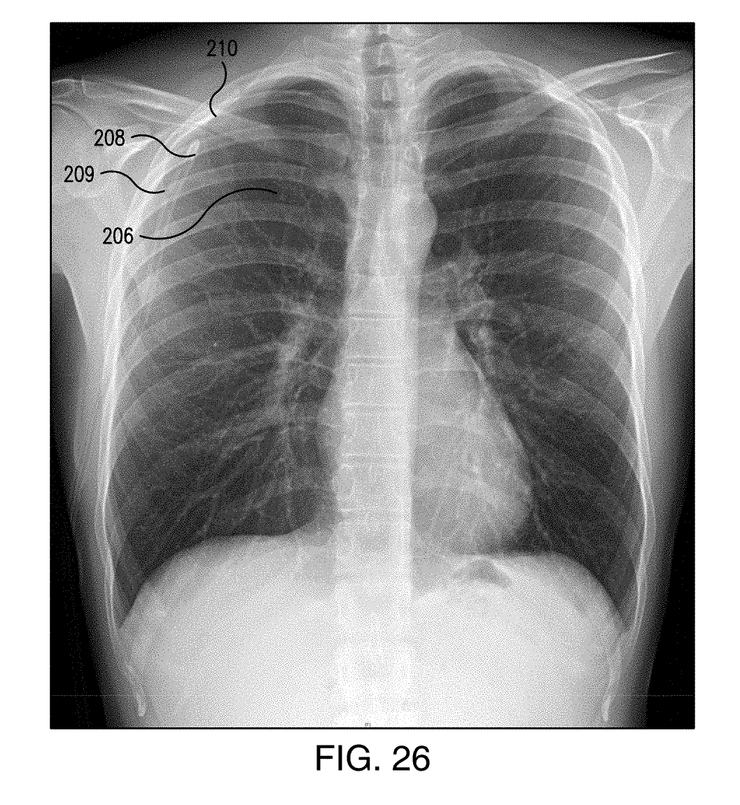

FIG. 26 is a follow-up chest X-ray of the patient of FIG. 25 right after switching to a digital chest drainage system which has a regulated susceptibility to air retraction, showing no pneumothorax.

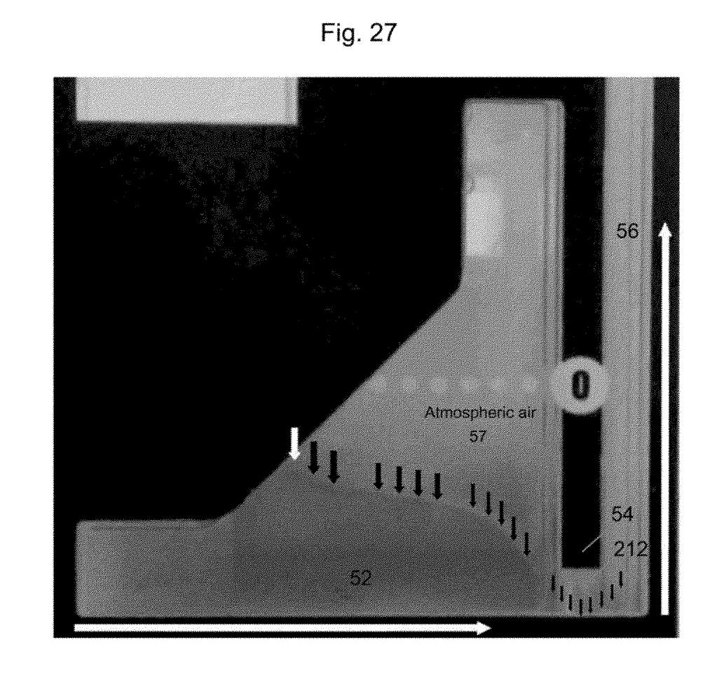

FIG. 27 is a photograph illustrating air retraction through an integrated, 3-bottle, UWSD system.

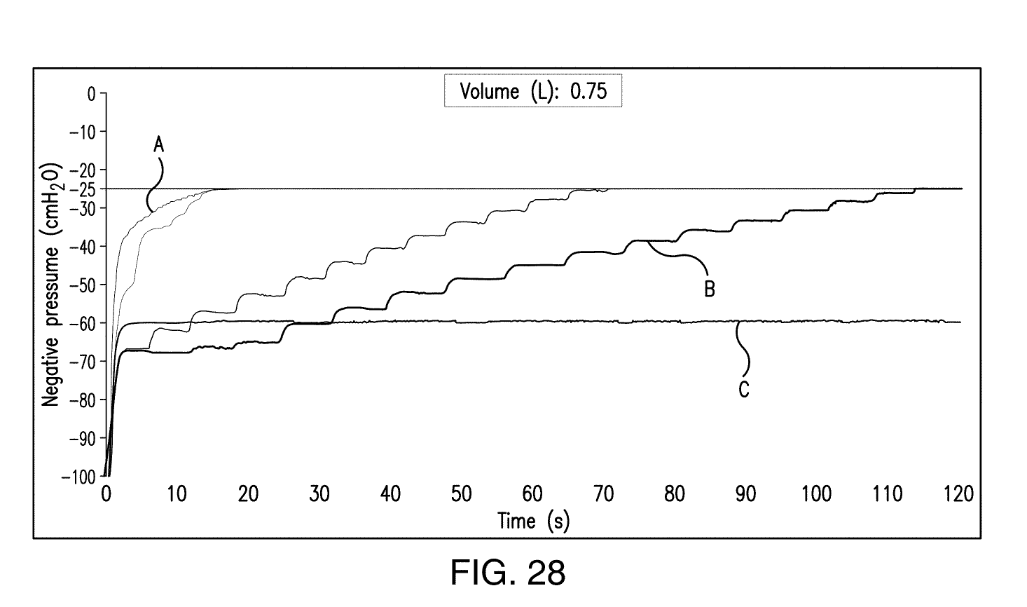

FIG. 28 is a graphical comparison of several chest drainage systems for handling a high negative intrapleural pressure.

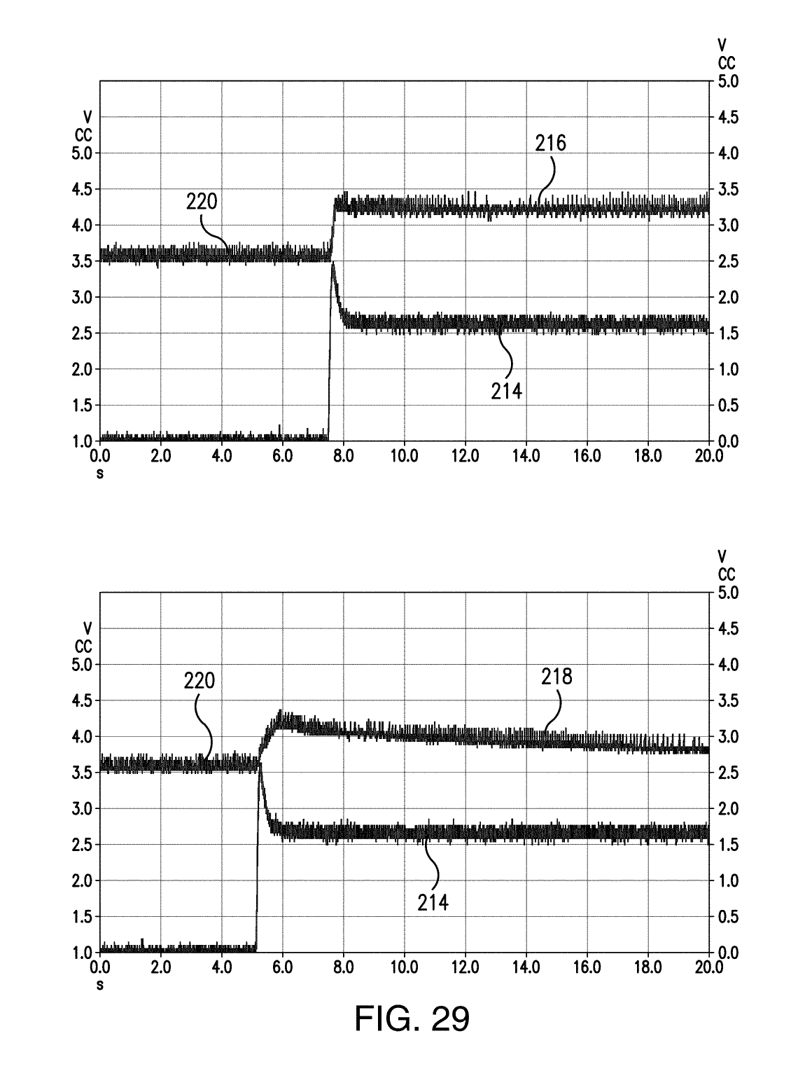

FIG. 29 is a graphical comparison of two chest drainage systems for handling a bronco-pulmonary air leak and re-establishing a physiologically negative intrapleural pressure.

FIG. 30 is a sketch of an integrated, 3-bottle, UWSD system, as known from the prior art.

Little progress has been done in the field of customized chest drainage therapy. There is still need for standardized treatments, which would then open the door to personalized algorithms. Patients are bearing the cost of this cultural and scientific heterogeneity, hence a paradigm shift is needed. The device, system and method disclosed below is well poised to provide doctors with key concepts: a) Not every chest drainage system works the same; b) Digital chest drainage systems are not necessarily better than “traditional” ones nor are they needed for every patient; c) Specific patients, such as those at high surgical risk (e.g. fragile lung tissue, prolonged healing time in long-time smokers), are the ones who would benefit the most from personalized therapeutic approaches.

Educational models of a phenomenon or activity allows users to rehearse behaviors and test equipment without placing clients or institutional resources at risk. The device, system and method disclosed herein answer this need by offering real-time analytics applicable by doctors for further academic research and clinical decision making.

Disclosed below are a device, system and method for characterizing a chest drainage apparatus. The device includes a source of both positive pressure and negative pressure, a conduit to provide the positive pressure and the negative pressure to the chest drainage apparatus and a sensor to detect a response of said chest drainage apparatus to either said positive pressure or said negative pressure. The system includes a device that controllably provides either a positive pressure or a negative pressure to the chest drainage apparatus and a sensor to record the effect of the positive pressure or the negative pressure to the drainage apparatus. The method includes the steps of (1) providing a source of both positive pressure and negative pressure; (2) controllably applying either the positive pressure or the negative pressure to the chest drainage apparatus; and (3) detecting a response from the chest drainage apparatus to the pressure application.

With reference to FIG. 1, the device 10 includes a circuit motherboard 12 populated with components shown within demarcation line 14. Some components, such as pressure transmitter 16 and laser sensor 18 are in communication with the motherboard 12, but are typically not located on the motherboard. The device 10 communicates with a chest drainage system being evaluated. Output, either vacuum pressure (a pressure less than ambient) or air flow (a pressure greater than ambient), is delivered to a connecting tube of the chest drainage system. Pump 40 generates an appropriate output for either vacuum pressure or for air flow.

A power source 20 is connected to an electrical outlet to receive standard AC current, such as 110 V/60 Hz AC (United States) or 220 V/50 Hz AC (Europe) is converted to 24 V DC current at transformer 22 and provided to device 10 components by power bus 24. A microprocessor 26 receives inputs from one or more of (dependent on evaluation being run) laser sensor electronics 28, pressure transmitter 16 and flow transmitter 30. The microprocessor outputs data to pressure control valve 32, a plurality of electrovalves 34 (EV0-EV6 being illustrated in FIG. 1) and data transmission port 38.

Data collected by the microprocessor 26 is transmitted by data bus 36 to a data transmission port 38. The data is then transmitted to a personal computer, mainframe computer, tablet, smart phone or other digital processing device to process and save the information received. Data transmission may be into a local area network (LAN), the internet, or any other suitable private or public network. Transmission to the digital processing device may be through a data cable or by wireless communication.

FIG. 2 is a photograph of the device 10 showing a control panel 42 and external components including a vacuum chamber 98 and laser sensor 18. Data communication line 44 connects the laser sensor to laser sensor electronics (28 in FIG. 1). Pneumatic tube 46 connects the vacuum chamber 98 to other pneumatic components of the device 10 as disclosed below. FIG. 3 is a photograph of the vacuum chamber 98. One suitable vacuum chamber has a volume of 0.75 L, although other capacity vacuum chambers are equally suitable. The vacuum/positive pressure pump (40 in FIG. 1) draws a negative pressure on the vacuum chamber 98 until a desired negative pressure is achieved. As disclosed below, in some evaluation procedures, the pump is then isolated from the test and a vacuum applied to an integrated, 3-bottle, UWSD system via the vacuum chamber.

FIG. 4 is a photograph of laser sensor 18 mounted to a support 48 and aligned with the base of the conduit 56 of an integrated, 3-bottle, UWSD system as used when characterizing a chest drainage system for susceptibility to air retraction. In the integrated, 3-bottle, UWSD system a water-seal chamber 52 in combination with a weir 54 is considered in the art to function as a “one-way” valve for air. Air may flow from an intake chest tube that is connected to the connecting tube, through the water of the water-seal chamber (“bubbles”), to the atmospheric side 57 of the water-seal chamber, but should normally not back-flow after chest tube placement during inspiration especially for a patient having a base highly negative intrapleural pressure, such as due to lung fibrosis. The size and quantity of air bubbles in the conduit 56 is a function of the pressure, which may be positive or negative, generated by the pump (40 in FIG. 1) and applied to the chest drainage system under evaluation by a pneumatic line. An intermittent or continuous air flow (“bubbles”) around the weir 54 is an indication that the chest drainage system under test is subject to a risk of back-flow.

The laser sensor 18 functions as an air detector, detecting air bubbles or air pockets and electrically communicating the data to laser sensor electronics (28 in FIG. 1) which then communicates with the microprocessor (26 in FIG. 1) via laser sensor cable 58 that is an electrical line. The laser sensor 18 interacts with the water-seal chamber 52 of the UWSD system. The support 48 holds the laser sensor 18 at a fixed position relative to the portion of conduit 56 filled with water at atmospheric pressure. Fixture 60 enables the laser sensor to be adjusted both in height and angle relative to conduit 56.

One suitable laser sensor 18 is the Series IL Intelligent—L Laser Sensor manufactured by Keyence Corporation of Itasca, Ill., USA. This sensor varies laser power according to reflectance of the target. Since reflectance of an air bubble will be different than reflectance of a liquid, measurement of the laser power will provide an accurate detection of air bubbles that pass across the detector field.

FIG. 5 is a schematic of the control panel 42 shown in the photograph of FIG. 2 and FIG. 6 is a closer photograph of that control panel. The control panel is accessible when the top cover of the device is open. Electrical connection 62 is for the laser sensor (18 in FIG. 1) and data transmission port 38 is for connection to an external digital computing system. Pneumatic access 64 connects to the vacuum chamber (98 in FIG. 1) and positive/negative pressure access 66 connects to the connecting tube of the chest drainage system under test. Fuses are connected to various components to be protected from overcurrent. As shown, fuse 68 is for vacuum power, 70 is for the pressure regulator, 72 is for the flow sensor, 74 is for the laser sensor, 76 is for 24 V DC power and 78 is for power status. Indicator lights 80 indicate the status of those components and outlet 82 is to receive a power cord from an external A/C source.

The function of the device will be more apparent from the descriptive schemas for three tests available utilizing the device as described below. These three tests are:

A) Reverse Air Flow (RAF)—Laser sensor and custom support are external accessories. Air compressor, Flow Restrictor and Pressure Transmitter may be external accessories;

B) High Sustained Negative Pressure—Vacuum chamber is an external accessory. Air Compressor and Pressure Transmitter may be external accessories; and

C) Air Package—Air Compressor and Pressure Transmitter may be external accessories.

“Traditional” chest drainage systems allow air to exit the pleural space, preventing reflux within the pleural space, by means of an interposed water-seal chamber (“one-way” valve). Certain patients, when some types of UWSD systems are used, are able to retract atmospheric air and fluids in the pleural space during inspiration. Utilizing the device described hereinabove and the method illustrated in FIGS. 7-10, the susceptibility of a particular chest drainage system to air retraction is determined.

FIG. 7 illustrates the device 10 connected to an integrated, 3-bottle, UWSD system collection chamber by its connecting tube. Electrovalve 86 (EV6 in FIG. 1) is initially open and the UWSD system is open to the atmosphere. At FIG. 8, components to the left (“left” and “right” are exemplary to illustrate sections relative to demarcation line and not intended to be limitations) of demarcation line 14 are set to a desired pressure, typically between −10 cm H₂O and −100 cm H₂O as follows: An external computer, with appropriate software (not shown or claimed) sends a command to the microprocessor 26. The vacuum pump 40 is actuated. The pressure transmitter 16 measures the generated pressure which is then regulated by the pressure control valve 32 and set to a desired test pressure. In technical terms, this is called closed-loop system regulation. Electrovalves 88 (EV2 in FIGS. 1) and 90 (EV3 in FIG. 1) are closed and electrovalve 92 (EV5 in FIG. 1) is opened so that the vacuum reaches the pressure transmitter 16 but not the UWSD system 50 that remains open to the atmosphere via electrovalve 86.

At FIG. 9, electrovalves 88, 90 are opened and electrovalve 86 is closed hence applying the desired test pressure on the UWSD system 50. Since the desired test pressure is less than atmospheric pressure, a vacuum is applied on the UWSD system 50 and air may be drawn through the chest drainage system being tested. The output of the chest drainage system under test is a series of air bubbles 94 in conduit 56. The laser sensor 18 detects the air bubbles and transmits the data via data communication line 44 to the laser sensor electronics (28 in FIG. 1) for transmission to the microprocessor (26 in FIG. 1).

At FIG. 10, electrovalves 88, 90 are closed and electrovalve 86 opened placing the UWSD system 50 at atmospheric pressure causing the air bubbles (94 in FIG. 9) in the conduit 56 to flow back in the water-seal chamber 52.

Newly designed chest drainage systems use digital electronics to maintain a continuous negative pressure differential. With reference to FIGS. 11-14, high negative intrapleural pressure swings (peaks) as well as sustained high negative intrapleural pressure (average) can be encountered after routine thoracic surgical procedures particularly when some newly designed digital chest drainage systems are used. This is because there may be no vacuum release mechanism (i.e. high-negative pressure valve) built in or because such mechanism does not work quickly enough to buffer a given amount of vacuum. This can happen also with certain “traditional” chest drainage systems (UWSD systems or dry seal).

With reference to FIG. 11, the device 10 is used to characterize digital chest drainage system 96. In start-up mode, the digital drainage system 96 is powered on and isolated from the device by closing electrovalves 88, 90. Next, as shown in FIG. 12, the vacuum chamber 98 is set to a desired negative pressure, typically between −30 cm H₂O and −100 cm H₂O as follows: the external computer, with appropriate software sends a command to the microprocessor (26 in FIG. 1). Vacuum pump 40 is actuated. Pressure transmitter 16 measures the generated pressure. The pressure control valve 32 regulates this generated pressure and sets it to a desired test pressure. In technical terms, this is called closed-loop system regulation. Electrovalve 92 is a three-way valve. First port 100 and third port 104 are open so that the vacuum reaches the pressure transmitter 16. Second port 102 is closed preventing the vacuum from reaching the digital chest drainage system 96 which remains isolated from the system.

Referring to FIG. 13, electrovalves 88, 90 are opened and electrovalve 106 (EV4 in FIG. 1) is closed releasing the vacuum into the digital drainage system 96 and monitoring through pressure transmitter 16 how long and how smoothly the digital chest drainage system 96 lowers the vacuum generated in the vacuum chamber 98 to a target pressure (set through the software), which is a more positive, but still sub-atmospheric pressure, usually between −10 cm H₂O and −30 cm H₂O. When the target pressure is reached, the vacuum pump 40 is turned off with the digital chest drainage system 96 stable at its own target pressure (FIG. 14).

FIGS. 15-18 illustrate an air package test used to simulate a bronco-pulmonary air leak from a patient and to determine the capability of a particular “traditional” or digital chest drainage system to evacuate such air leak from the pleural space, thereby reestablishing a physiologically negative pressure. As shown in FIG. 15, at the start of a test, the chest drainage system, either UWSD 50 or digital 96 (or dry-sealed or hybrid) is isolated from the device 10 because electrovalves 88, 90 are closed. Also closed is the first port 100 of electrovalve 92. As shown in FIG. 16, pump 40 set to deliver positive pressure is actuated and electrovalves 88, 90 are opened starting a flow of air. First port 108 of electrovalve 90 remains closed isolating the chest drainage system 50, 96 from the flow of air. Second port 110 of electrovalve is opened to atmosphere allowing the air flow to escape (vent 112).

FIG. 17 shows the test portion of the air test package. Second port 110 of electrovalve 90 is closed and first port 108 is opened enabling air flow to the chest drainage system 50, 96 generating a flow of bubbles to the UWSD collector 50 and a positive pressure to the digital device 96. The device 10 monitors, through pressure transmitter 16, how long and how smoothly the chest drainage system 50, 96 evacuates air flow generated and reaches the target pressure (set through the software). At the conclusion of the test, as shown in FIG. 18, the chest drainage system 50, 96 is isolated from the air flow by closing first port 108 of electrovalve 90 and opening the second port 110 to vent 112.

FIG. 19 schematically illustrates a device 150 for characterizing a chest drainage system in accordance with a second embodiment. The device 150 utilizes fewer electrovalves than the device 10 described above. Further, the vacuum/positive pressure pump (40 in FIG. 1) is replaced with a combination of an air compressor and a venturi to generate an air flow or a vacuum as need for the reverse air flow, high negative pressure and air package characterizations.

A control panel 152 for the device 150 is shown in FIG. 20. Control panel 152 has ports 154, 156, 158, 160, 162, 164, 166 and 168 for connecting pneumatic lines to the device and electrical connectors 170, 172, 174 and 176 for power and data. In an exemplary embodiment, shown in FIG. 20, electrical connector 170 is for receipt of AC power. Electrical connector 172 connects to the laser sensor and electrical connector 174 connects to the pressure transmitter. Electrical connector 176 is for the transfer of data to and from the device.

Port 154 connects to the air compressor and port 168 connects to the connecting tube of the chest drainage apparatus under test. Connections to the other ports varies according to the test being performed. FIG. 21 illustrates a pneumatic configuration for a reverse air flow characterization. Air compressor 178 is connected to the venturi 180 for generating a vacuum. Between the air compressor 178 and the venturi 180 are pressure regulator 202 and pressure control valve 204 that in combination regulate the air flow into the venturi thereby regulating the generated pressure. The air compressor creates an air flow at a pressure of between about 3 Bar and 6 Bar. A pneumatic line 182 connects ports 156 and 158 so air flow from the compressor 178 enters venturi 180 generating a vacuum on pneumatic line 184 that connects to a first three way valve 186 and a second three way valve 188 via pneumatic lines 190, 192. Closing the second three way valve 188 isolates the collection chamber 50 of the integrated, 3-bottle, UWSD system that is under test when connected to the device by way of connecting tube 84. Pressure is measured by pressure transmitter 16. When the desired pressure is obtained, second three way valve 188 is opened applying a vacuum on the collection chamber 50 for the reverse air flow characterization.

FIG. 22 shows the pneumatic configuration of the second device 150 (FIG. 19) for high negative intrapleural pressure characterization. The combination of air compressor 178 and venturi generates a vacuum in pneumatic line 190 as described above. The first three way valve 186 is open enabling the pressure transmitter 16 to measure the vacuum. Second three way valve 188 has a first port 194 open so that the vacuum is collected in vacuum chamber 98. Second port 196 and third port 198 of second three way valve 188 are closed isolating the digital drainage system 96 from the vacuum. When the digital drainage system is to be characterized, the first three valve 186 is closed isolating the vacuum generating components 178, 180. Third port 198 of the second three way valve 188 is then opened applying a vacuum on the digital drainage system 96 via pneumatic line 200. FIG. 23 shows the pneumatic configuration for the air package characterization.

Venturi 180 is isolated from the system and air flow generated by the air compressor is regulated by pressure regulator 202, pressure control valve 204 and flow transmitter 30. When the second port 196 and third port 198 of second three way valve 188 are open and first port 194 is closed, the device under test, 50, 96 is isolated from the system and the air flow escapes through vent port 205. Closing second port 196 and opening first port 194 enable air flow to the device under test 50, 96.

FIG. 24 is sequential, scaled, graphical comparison of four integrated, 3-bottle, UWSD systems for susceptibility to air retraction. The vertical axis represents negative pressure in cm H₂O while the horizontal axis represents time in seconds. The filled circles represent the time from when the vacuum is applied (time 0) until the first air bubble is detected. A moderate amount of time, for example 15-20 seconds at −30 cm H₂O is preferred over shorter or longer times because if the air comes back too soon, air flows back to the chest tube, potentially at each inspiration, and therefore a pulmonary air leak cannot be differentiated from atmospheric air retracted because of sub-optimal apparatus geometry. If the time is too long, the vacuum is not buffered, therefore accumulating into the pleural space, potentially damaging intrathoracic structures.

FIG. 25 and FIG. 26 are chest X-Rays from the same patient showing a clinical impact of reverse air flow. Referring to FIG. 26, there are medical conditions requiring a lung 206 to be reattached to a chest wall 209. Subsequent to that reattachment, distal end 208 of a chest tube is inserted into the pleural space 210 (virtual space) to drain air and fluids. Referring to FIG. 25, if the chest drainage system permits reverse air flow, the lung 206 may detach from the chest wall 209 as evidenced by a large volume of air now filling the pleural space 210.

FIG. 27 shows a UWSD system characterized as prone to reverse air flow. Vacuum is applied at the conduit 56 and retracts air back by bending the surface of the water contained into the water-seal chamber 52.

To evaluate handling of high negative pressure, FIG. 28 is a graphical representation of the recovery time and pattern of recovery of four digital chest drainage systems and one hybrid chest drainage system set to operate at a continuous negative pressure of −20 cm H₂O. An exemplary device (10 in FIG. 1) sets a 0.75 L vacuum chamber to a test pressure of −100 cm H₂O as shown at time 0 in FIG. 13. When the device 10 works at phase 2 (FIG. 13) the time and smoothness by which the chest drainage system lowers the test pressure to the target pressure of −25 cm H₂O (set through the software) is recorded. As shown in FIG. 28, some digital drainage systems recover quickly and smoothly (Reference line A), others take a considerably longer time and recover in a step-like fashion (Reference line B), while the hybrid never recovers (Reference line C). A digital chest drainage system most closely corresponding to reference line A is preferred.

To evaluate air packages, FIG. 29 is a graphical representation of the recovery time and pattern of recovery from a flow of air for two digital chest drainage systems. Bottom line 214 illustrates an air flow generated by the device 10. Top lines 216, 218 illustrates pressure in the chest drainage system under test. Line 216 remains at an elevated pressure above the base line 220. Line 218 shows a gradual recovery to the base line 220 and is a preferred system.

Reverse air flow, high negative intrapleural pressure, and low rates of air evacuation for air packages can lead to a higher rate of post-operative complications such as prolonged air leaks, causing prolonged chest tube duration (increased pain, immobility and risk for infections), prolonged length of hospital stay (costs), incomplete parenchymal re-expansion, subcutaneous emphysema and reoperations.

In particular, unregulated reverse airflow may affect a physician's decision-making process in judging the origin of air bubbles. Are the air bubbles from an unhealed lung parenchyma and, hence, true air leaks? Or are the air bubbles from the outside and, hence, fake air leaks? High negative intrapleural pressure is likely to set the stage for higher postoperative complications, and even mortality, in high-risk patients. Air packages in the context of prolonged air evacuation time would generate back-pressure with lung collapse and subcutaneous emphysema which would neutralize the intent of certain surgical techniques i.e. pleurodesis for pneumothorax, since the adhesion of the visceral to the parietal pleura would be disrupted.

Utilizing the data generated above, physicians would gain insight into the predicted clinical performances of any given chest drainage system through a quick assessment of their technical features. Pre- and post-operative patient parameters (input) can indeed be given to the device to obtain a representation (output) of how these technical features can impact how physicians care for their patients.

Physicians could therefore base their clinical decision making on laboratory data integrated with patient-specific, real-time, parameters. They could for example rationally chose the chest drainage system whose characteristics are in line with pre-operative clinical expectations or plan on how to handle a potential postoperative complication.

These unbiased data could be shared among physicians thereby fostering good practice by disseminating and in depth technical understanding clinically applicable. In fact, many surgeons are often exposed to only one or two models bought by hospital administrators (price-dependent criteria) and may be unfamiliar—if not completely unaware—with the technical features and clinical implications of other models.

The system could also be adopted by companies who manufacture chest drainage companies to guide R&D and methodically benchmark their products.

A chest drainage system capable to tailor chest drainage therapy to individual patients is based on the data developed by the device and system described above. Preoperative patient parameters are programmed into the invention providing doctors with direct control (beside regular feedback) on the patient predicted outcome. By knowing the performances of any given chest drainage system, doctors could anticipate well-known complications, providing instantly better patient care and therefore faster recovery and reduced costs for the hospital. By integrating bench data with patient specific parameters, an algorithm to program the circuit motherboard and make it capable to react in real time to changes in each patient conditions, such as air leaks and intrapleural pressures, is being developed. Different sizes (volumes) or air leaks and changes in intrapleural pressure are tested and validated using the testing system. The acquire knowledge is returned to the patient's bedside resulting in improved patient outcomes and providing a basis for better marketable chest drainage systems.

Based on integration between real-time chest drainage system performances and programmable patient characteristics, doctors would be able to benchmark any model of chest drainage system prior to surgery. Choosing, on a case-by-case basis, the optimal chest drainage therapy would present a custom approach to chest drainage management.

1. A device for characterizing a chest drainage apparatus, comprising:
a source of both positive pressure and negative pressure, relative to ambient pressure;
a conduit to provide said positive pressure and said negative pressure to the chest drainage apparatus; and
a sensor to detect a response of said chest drainage apparatus to either said positive pressure or said negative pressure.

2. The device of claim 1 wherein said source is a pump effective to selectively generate either a positive pressure or a negative pressure.

3. The device of claim 2 wherein a plurality of electrovalves selectively transmit either the positive pressure or the negative pressure to the conduit.

4. The device of claim 1 wherein an air compressor is in communication with a venturi whereby positive pressure is provided by direct flow from said air compressor and negative pressure is generated when the output of the air compressor flows through said venturi.

5. The device of claim 4 wherein a plurality of pneumatic lines selectively transmit either the positive pressure or the negative pressure to the conduit.

6. The device of claim 1 wherein the sensor includes a light beam and a light detector.

7. The device of claim 6 wherein a laser generates said light beam and said light detector measures reflectance of said light beam.

8. The device of claim 7 wherein said laser sensor is mounted in a support effective to fix both elevation and angle.

9. The device of claim 3 further including a vacuum chamber.

10. The device of claim 9 wherein said electrovalves isolate said conduit from said source whereby a desired vacuum is drawn on said vacuum chamber.

11. The device of claim 5 further including a vacuum chamber.

12. The device of claim 11 wherein said pneumatic lines isolate said conduit from said source whereby a desired vacuum is drawn on said vacuum chamber.

13. The device of claim 10 wherein said sensor detects pressure as a function of time.

14. The device of claim 13 wherein said sensor identifies time 0 as when said desired vacuum is applied to said chest drainage apparatus.

15. The device of claim 3 wherein said electrovalves isolate said source of positive pressure from said conduit.

16. The device of claim 16 wherein said sensor identifies a volume of air flow as a function of time.

17. The device of claim 16 wherein said sensor identifies time 0 as when said volume of air flow is applied to said chest drainage apparatus.

18. A system to characterize a chest drainage apparatus, comprising:
a device that controllably provides either a positive pressure or a negative pressure to said chest drainage apparatus; and
a sensor to record the effect of said positive pressure or said negative pressure to said chest drainage apparatus.

19. The system of claim 18 wherein said chest drainage apparatus includes a connecting tube for pneumatically joining a chest drainage tube to a collection chamber and said device is pneumatically joined to said connecting tube in lieu of said chest drainage tube whereby said device communications with said collection chamber.

20.-32. (canceled)

33. A method to characterize a chest drainage system, comprising the steps of:
providing a source of both positive pressure and negative pressure relative to ambient pressure;
controllably applying either said positive pressure or said negative pressure to said chest drainage apparatus; and
detecting a response from said chest drainage apparatus to said pressure application.

34.-41. (canceled)
